(19) United States
(12) Patent Application Publication
(10) Pub. No.: US 2024/0335484 A1
(43) Pub. Date: Oct. 10, 2024

(54) POSTBIOTIC

(71) Applicant: SOCIETE DES PRODUITS NESTLE S.A., Vevey (CH)

(72) Inventor: KATJA JOHNSON, Konolfingen (CH)

(21) Appl. No.: 18/294,358

(22) PCT Filed: Aug. 18, 2022
(86) PCT No.: PCT/EP2022/073082

§ 371 Date: Feb. 1, 2024

(30) Foreign Application Priority Data
Aug. 19, 2021 (EP) 21192228.1

(51) Int. Cl. A61K 35/745; A61P 37/06

(52) U.S. Cl. CPC A61K 35/745; A61P 37/06

(57) ABSTRACT

The present invention provides a Bifidobacterium lactis supernatant for use in enhancing expression of anti-inflammatory cytokines and/or reducing expression of pro-inflammatory chemokines in the gastrointestinal tract of a subject having or at risk of an overactive immune system disorder. The present invention also provides a Bifidobacterium lactis supernatant for use in enhancing expression of IL-10 in the gastrointestinal tract of a subject having or at risk of an IL-10 mediated disease. The present invention also provides a Bifidobacterium lactis supernatant for use in treating or preventing an IL-10 mediated disease by enhancing expression of IL-10 in the gastrointestinal tract of a subject.

### FIELD OF THE INVENTION

The present invention relates to postbiotics and their use in enhancing expression of anti-inflammatory cytokines (e.g. IL-10) and/or reducing expression of pro-inflammatory chemokines. The present invention also relates to the use of postbiotics in treating an overactive immune system disorder (e.g. an IL-10 mediated disease).

### BACKGROUND TO THE INVENTION

Anti-inflammatory cytokines act along with specific cytokine inhibitors and soluble cytokine receptors to regulate the human immune response. Major anti-inflammatory cytokines include interleukin (IL)-1 receptor antagonist, IL-4, IL-6, IL-10, IL-11, and IL-13 (Opal, S. M. and DePalo, V. A., 2000. Anti-inflammatory cytokines. Chest, 117(4), pp. 1162-1172).

Interleukin (IL)-10 is an important anti-inflammatory cytokine produced by many cell populations. The main biological function of IL-10 is the limitation and termination of inflammatory responses and the regulation of differentiation and proliferation of several immune cells such as T cells, B cells, natural killer cells, antigen-presenting cells, mast cells, and granulocytes (Asadullah, K., et al., 2003. Pharmacological reviews, 55(2), pp. 241-269).

Dysregulation of anti-inflammatory cytokines such as IL-10 can result in chronic systemic inflammation and an overactive immune system. For example, an IL-10 deficiency can result in severe dysregulation of the immune system and can enhance inflammatory response to microbial challenge and lead to development of inflammatory bowel disease (IBD) and a number of autoimmune diseases (Iyer, S. S. and Cheng, G., 2012. Critical Review in Immunology, 32(1)).

However, existing solutions to enhancing the expression of anti-inflammatory cytokines (e.g. IL-10) have a number of drawbacks. Systemic IL-10 treatment is typically not very effective in inducing clinical remission and is associated with considerable side effects. Although some probiotic strains have been shown to enhance IL-10 expression, probiotics need to be kept alive, limiting their application (de Moreno de LeBlanc, A., et al., 2011. ISRN 2011, Article ID 892971).

Thus, there is a demand for alternative agents and compositions which can enhance the expression of anti-inflammatory cytokines (e.g. IL-10) in the gastrointestinal tract of a subject.

### SUMMARY OF THE INVENTION

Bifidobacterium animalis subsp. lactis (also known as Bifidobacterium lactis or B. lactis) supernatant has surprisingly been found to exhibit immune-modulatory effects superior in several aspects to those of the B. lactis probiotic, which is well-documented in the literature for its immune-modulatory effects in various clinical trials.

Bifidobacterium lactis supernatant strongly increased the secretion anti-inflammatory cytokines IL-6 and IL-10 compared to B. lactis. Moreover, Bifidobacterium lactis supernatant decreased the secretion of pro-inflammatory chemokine IL-8. Of particular note, Bifidobacterium lactis supernatant more than doubled the secretion of IL-10.

In one aspect, the present invention provides a Bifidobacterium lactis supernatant.

In another aspect, the present invention provides use of a Bifidobacterium lactis supernatant as an immunomodulator.

In another aspect, the present invention provides use of a Bifidobacterium lactis supernatant as an immunosuppressant.

In another aspect, the present invention provides a Bifidobacterium lactis supernatant for use in enhancing expression of anti-inflammatory cytokines and/or reducing expression of pro-inflammatory chemokines in the gastrointestinal tract of a subject having or at risk of an overactive immune system disorder. In some embodiments, the Bifidobacterium lactis supernatant enhances expression of IL-10 in the gastrointestinal tract of the subject and/or the overactive immune system disorder is an IL-10 mediated disease.

In another aspect, the present invention provides a Bifidobacterium lactis supernatant for use in enhancing expression of IL-10 in the gastrointestinal tract of a subject having or at risk of an IL-10 mediated disease.

In another aspect, the present invention provides a Bifidobacterium lactis supernatant for use in treating or preventing an IL-10 mediated disease by enhancing expression of IL-10 in the gastrointestinal tract of a subject.

Any suitable Bifidobacterium lactis strain may be used in the present invention. In some embodiments, the Bifidobacterium lactis is selected from: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010. In some embodiments, the Bifidobacterium lactis is a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446. In some embodiments, the Bifidobacterium lactis is Bifidobacterium animalis subsp. lactis CNCM I-3446.

The Bifidobacterium lactis supernatant may be obtained or obtainable by culturing Bifidobacterium lactis in a suitable culture media. In some embodiments, the culture medium comprises sugar and yeast extract, and, optionally, sodium ascorbate and/or polysorbate. In some embodiments, the culture media comprises: (i) about 1 wt % to about 6 wt %, or about 2 wt % to about 4 wt % sugar; (ii) about 1 wt % to about 10 wt %, or about 1 wt % to about 6 wt %, or about 2 wt % to about 4 wt % yeast extract; (iii) about 0 wt % to about 0.5 wt %, or about 0.1 wt % to about 0.2 wt % sodium ascorbate; and/or (iv) about 0 wt % to about 1 wt %, or about 0 wt % to about 0.3 wt % polysorbate. In some embodiments, the pH is controlled at a pH of from about 5 to about 7, a pH of from about 5.5 to about 6.5, or a pH of about 6. The Bifidobacterium lactis may be cultured under any suitable conditions. In some embodiments, the Bifidobacterium lactis is cultured until stationary phase is reached. In some embodiments, the Bifidobacterium lactis is cultured under anaerobic conditions.

Any suitable processing steps may be used to obtain a Bifidobacterium lactis supernatant. The Bifidobacterium lactis supernatant may be obtained or obtainable by removing all or substantially all the Bifidobacterium lactis cells from a Bifidobacterium lactis fermentate. In some embodiments, the Bifidobacterium lactis cells are removed by centrifugation.

In some embodiments, the Bifidobacterium lactis supernatant is pasteurised. In some embodiments, the Bifidobacterium lactis supernatant is dried. In some embodiments, the Bifidobacterium lactis supernatant is dried by spray-drying. In some embodiments, the Bifidobacterium lactis supernatant is spray-dried with a carrier material selected from one or more of: oat fibre, maltodextrin, acacia gum, starch and inulin. In some embodiments, the Bifidobacterium lactis supernatant is spray-dried with acacia gum. In some embodiments, the Bifidobacterium lactis supernatant and carrier material are mixed in a total solids ratio of from about 1:3 to about 2:1 (carrier:supernatant dry solids), preferably wherein the total solids ratio is about 1:1 (carrier:supernatant dry solids).

The Bifidobacterium lactis supernatant may be provided in any suitable form. In some embodiments, the Bifidobacterium lactis supernatant is in a form suitable for oral administration. In some embodiments, the Bifidobacterium lactis supernatant is in the form of a supplement or a nutritional composition. In some embodiments, the Bifidobacterium lactis supernatant is in the form of a capsule or a tablet.

During culturing the Bifidobacterium lactis will consume nutrients in the culture media (e.g. sugar and amino acids) and enrich the culture media with various soluble factors (e.g. organic acids and other metabolites). In some embodiments, compared to Bifidobacterium lactis culture media the Bifidobacterium lactis supernatant has: (i) a decreased concentration of total sugar; (ii) an increased concentration of total acids; and/or (iii) a decreased concentration of total amino acids. In some embodiments, compared to Bifidobacterium lactis culture media: (i) the concentration of total sugar has decreased by at least about 50%, at least about 60%, at least about 70%, at least about 80%, or at least about 90%; (ii) the concentration of total acids has increased by at least about 70%, by at least about 80%, or by at least about 90% of the concentration decrease in total sugars; and/or (iii) the concentration of total amino acids in the media has decreased by at least about 0.1 wt %, by at least about 0.2 wt %, or by at least about 0.3 wt %.

In some embodiments, before pasteurisation the Bifidobacterium lactis supernatant has a viable cell count of from about 1×10⁷ to about 1×10⁹ cfu/ml. In some embodiments, the Bifidobacterium lactis supernatant comprises: (i) about 4 wt % or less, about 3 wt % or less, about 2 wt % or less, about 1 wt % or less, or about 0.5 wt % or less total sugars; (ii) about 0.5 wt % or more, about 1 wt % or more, about 1.5 wt % or more, or about 2 wt % or more total acids; and/or (iii) about 3.5 wt % or less, about 2 wt % or less, about 1 wt % or less, about 0.8 wt % or less, or about 0.6 wt % or less total amino acids. In some embodiments, the Bifidobacterium lactis supernatant has a pH of from about 5 to about 7, from about 5.5. to about 6.5, or of about 6, preferably wherein the Bifidobacterium lactis supernatant has a pH of about 6.2.

The Bifidobacterium lactis supernatant may be used in combination with any other suitable agent or composition. In some embodiments, the Bifidobacterium lactis supernatant is used in combination with one or more probiotics, prebiotics, or synbiotics, preferably wherein the Bifidobacterium lactis supernatant is used in combination with one or more probiotics. In some embodiments, the Bifidobacterium lactis supernatant is used in combination with a Bifidobacterium lactis probiotic, preferably wherein the probiotic Bifidobacterium lactis is the same as the Bifidobacterium lactis from which the supernatant is derived.

The Bifidobacterium lactis supernatant may enhance the expression of one or more anti-inflammatory cytokines in the gastrointestinal tract of a subject. In some embodiments, the Bifidobacterium lactis supernatant enhances the expression of IL-6 and/or IL-10. In some embodiments, the Bifidobacterium lactis supernatant enhances the expression of IL-10.

The Bifidobacterium lactis supernatant may reduce the expression of one or more pro-inflammatory chemokines in the gastrointestinal tract of a subject. In some embodiments, the Bifidobacterium lactis supernatant reduces the expression of CXCL10 and/or IL-8. In some embodiments, the Bifidobacterium lactis supernatant reduces the expression of IL-8.

The Bifidobacterium lactis supernatant may be administered to any subject in need thereof. In some embodiments, the subject is human. In some embodiments, the subject has or is at risk of an overactive immune system disorder. In some embodiments, the subject has or is at risk of an IL-10 deficiency.

The Bifidobacterium lactis supernatant may be used to treat or prevent any suitable overactive immune system disorder or IL-10 mediated disease. In some embodiments, the IL-10 mediated disease is selected from: an inflammatory bowel disease (IBD), an allergic disease, dermatitis, an autoimmune disease, an infection-associated immunopathology, colorectal cancer, impaired bone healing, and atherosclerosis. In some embodiments: (i) the IBD is Crohn's disease (CD) or ulcerative colitis (UC); (ii) the allergic disease is allergic asthma or a food allergy; (iii) the dermatitis is atopic dermatitis or contact dermatitis; (iv) the autoimmune disease is selected from: systemic lupus erythematosus, rheumatoid arthritis, multiple sclerosis, psoriasis, ankylosing spondylitis, and Guillian-Barre syndrome; and/or (v) the infection is selected from: a protozoan infection, a bacterial infection, a nematode infection, a viral infection, and a fungal infection.

In another aspect, the present invention provides a supplement comprising a Bifidobacterium lactis supernatant. The supplement may be in the form of a capsule or a tablet.

In another aspect, the present invention provides a nutritional composition comprising a Bifidobacterium lactis supernatant.

The Bifidobacterium lactis supernatant present in the supplement or nutritional composition may be any Bifidobacterium lactis supernatant according to the present invention.

The Bifidobacterium lactis supernatant may be present in combination with any other suitable agent or composition. In some embodiments, the supplement or nutritional composition comprises one or more probiotics, prebiotics, or synbiotics, preferably wherein the supplement or nutritional composition comprises one or more probiotics. In some embodiments, the supplement or nutritional composition comprises a Bifidobacterium lactis probiotic, preferably wherein the probiotic Bifidobacterium lactis is the same as the Bifidobacterium lactis from which the supernatant is derived.

In another aspect, the present invention provides a method of manufacturing a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant; and
(c) optionally, pasteurising the Bifidobacterium lactis supernatant.

The method preferably further comprises a step (d) of drying the Bifidobacterium lactis supernatant, preferably wherein the Bifidobacterium lactis supernatant is dried by spray-drying.

The Bifidobacterium lactis supernatant manufactured by the method of the present invention may be any Bifidobacterium lactis supernatant according to the present invention.

In another aspect, the present invention provides a method of manufacturing a supplement comprising a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant;
(c) optionally, pasteurising the Bifidobacterium lactis supernatant;
(d) drying the Bifidobacterium lactis supernatant to provide a Bifidobacterium lactis supernatant powder; and
(e) encapsulating, compressing and/or packaging the Bifidobacterium lactis supernatant powder to provide a supplement comprising the Bifidobacterium lactis supernatant.

The steps may be carried out by any suitable means. In some embodiments, the Bifidobacterium lactis supernatant is dried by spray-drying. In some embodiments, the Bifidobacterium lactis supernatant powder is encapsulated to provide a capsule comprising the Bifidobacterium lactis supernatant.

The supplement manufactured by the method of the present invention may be any supplement according to the present invention.

### DESCRIPTION OF DRAWINGS

FIG. 1—Effect of colonic batch suspensions on NF-κB activity of THP1-Blue™ cells

NF-κB activity levels were measured 6h after LPS treatment on the basolateral side of the Caco-2/THP1-Blue™ co-cultures after pre-treatment of the apical side for 24h with the colonic batch suspensions. The dotted line corresponds to the experimental control LPS+. Data are plotted as mean±SEM. (*) represents statistically significant differences between the control and treatment samples. (*)=p<0.05; (**)=p<0.01; (***)=p<0.001. BL=Bifidobacterium animalis subsp. lactis CNCM I-3446; SUP=supernatant derived from Bifidobacterium animalis subsp. lactis CNCM I-3446; low=low dose variant; D1=donor 1; D2=donor 2; D3=donor 3; D1-D3=average of all 3 donors.

FIG. 2—Effect of colonic batch suspensions on secretion of IL-6 and IL-10

(A) IL-6 and (B) IL-10. Cytokine levels were measured 6h after LPS treatment on the basolateral side of the Caco-2/THP1-Blue™ co-cultures after pre-treatment of the apical side for 24h with colonic batch suspensions. The dotted line corresponds to the experimental control LPS+. Data are plotted as mean±SEM. (*) represents statistically significant differences between the treatment and the control. (*)=p<0.05; (**)=p<0.01; (***)=p<0.001; (****)=p<0.0001. BL=Bifidobacterium animalis subsp. lactis CNCM I-3446; SUP=supernatant derived from Bifidobacterium animalis subsp. lactis CNCM I-3446; low=low dose variant; D1=donor 1; D2=donor 2; D3=donor 3; D1-D3=average of all 3 donors.

FIG. 3—Effect of colonic batch suspensions on secretion of CXCL10 and IL-8

(A) CXCL10 and (B) IL-8. Cytokine levels were measured 6h after LPS treatment on the basolateral side of the Caco-2/THP1-Blue™ co-cultures after pre-treatment of the apical side for 24h with colonic batch suspensions. The dotted line corresponds to the experimental control LPS+. Data are plotted as mean±SEM. (*) represents statistically significant differences between the treatment and the control. (*)=p<0.05; (**)=p<0.01; (***)=p<0.001; (****)=p<0.0001. BL=Bifidobacterium animalis subsp. lactis CNCM I-3446; SUP=supernatant derived from Bifidobacterium animalis subsp. lactis CNCM I-3446; low=low dose variant; D1=donor 1; D2=donor 2; D3=donor 3; D1-D3=average of all 3 donors.

FIG. 4—Cell experiment results for the treatment colonic batch suspensions normalized to the blank control colonic batch suspensions Grey shading intensity is proportional to the degree of positive changes compared to the control. Values=1 (no change from control); values >1 (treatment higher than control); values <1 (treatment lower than control).

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

All publications mentioned in the specification are herein incorporated by reference.

Bifidobacterium lactis Supernatant

In one aspect, the present invention provides a Bifidobacterium lactis supernatant.

As used herein, “supernatant” may refer to the spent or partially spent culture media in which cells have been cultured. Typically, all or substantially all the cells are removed from the culture media at the end of culturing. For example, a supernatant may be obtained or obtainable by a method comprising: (a) culturing cells in a culture media to provide a fermentate; and (b) removing all or substantially all the cells from the fermentate to provide a supernatant.

As used herein, a “Bifidobacterium lactis supernatant” may refer to a supernatant derived from a Bifidobacterium lactis culture. For example, a Bifidobacterium lactis supernatant may be obtained or obtainable by a method comprising: (a) culturing Bifidobacterium lactis in a culture media to provide a Bifidobacterium lactis fermentate; and (b) removing all or substantially all the Bifidobacterium lactis cells from the Bifidobacterium lactis fermentate to provide a Bifidobacterium lactis supernatant.

The Bifidobacterium lactis supernatant may be referred to as a postbiotic. As used herein, a “postbiotic” may refer to soluble factors (products or metabolic by-products), secreted by live bacteria, or released after bacterial lysis, such as enzymes, peptides, teichoic acids, peptidoglycan-derived muropeptides, polysaccharides, cell surface proteins, and organic acids (see e.g. Aguilar-Toalá, J. E., et al., 2018. Trends in Food Science & Technology, 75, pp. 105-114).

In one aspect, the present invention provides a postbiotic comprising or consisting of a Bifidobacterium lactis supernatant.

Bifidobacterium lactis Strains

Bifidobacterium lactis (also known as Bifidobacterium animalis subsp. lactis, NCBI:txid302911) is a gram-positive, anaerobic, rod-shaped bacterium of the Bifidobacterium genus which can be found in the large intestines of humans. Bifidobacterium animalis and Bifidobacterium lactis were previously described as two distinct species. Presently, both are considered B. animalis with the subspecies Bifidobacterium animalis subsp. animalis and Bifidobacterium animalis subsp. lactis (Masco, L., et al, 2004. International Journal of Systematic and Evolutionary Microbiology, 54(4), pp. 1137-1143).

Any suitable Bifidobacterium lactis strain may be used in the present invention. For example, any Bifidobacterium lactis strain which is known to have a probiotic effect may be used in the present invention. Such Bifidobacterium lactis strains will be well known to the skilled person.

Suitably, the Bifidobacterium lactis may be selected from: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

The Bifidobacterium lactis may be a Bifidobacterium animalis subsp. lactis having at least 99% sequence identity to any B. animalis subsp. lactis known to the skilled person. The Bifidobacterium lactis may be a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to any B. animalis subsp. lactis known to the skilled person. There are at least 103 genome assemblies publicly available.

The Bifidobacterium lactis may be a Bifidobacterium animalis subsp. lactis having at least 99% sequence identity to Bifidobacterium animalis subsp. lactis CNCM I-3446, Bifidobacterium animalis subsp. lactis BI12 (Accession no. CP004053), Bifidobacterium animalis subsp. lactis BLC1 (Accession no. CP003039), Bifidobacterium animalis subsp. lactis DSM10140 (Accession no. CP001606), Bifidobacterium animalis subsp. lactis V9 (Accession no. CP001892), Bifidobacterium animalis subsp. lactis BI-04 (Accession no. CP001515), Bifidobacterium animalis subsp. lactis Bi-07 (Accession no. NC_017867), Bifidobacterium animalis subsp. lactis B420 (Accession no. NC_017866), Bifidobacterium animalis subsp. lactis BB-12 (Accession no. CP001853), Bifidobacterium animalis subsp. lactis AD011 (Accession no. CP001213), Bifidobacterium animalis subsp. lactis HN019 (Accession no. CP031154), Bifidobacterium animalis subsp. lactis DN-173 010, Bifidobacterium animalis subsp. lactis ATCC 27536, or Bifidobacterium animalis subsp. lactis VTT E-012010.

The Bifidobacterium lactis may be a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446, Bifidobacterium animalis subsp. lactis BI12 (Accession no. CP004053), Bifidobacterium animalis subsp. lactis BLC1 (Accession no. CP003039), Bifidobacterium animalis subsp. lactis DSM10140 (Accession no. CP001606), Bifidobacterium animalis subsp. lactis V9 (Accession no. CP001892), Bifidobacterium animalis subsp. lactis BI-04 (Accession no. CP001515), Bifidobacterium animalis subsp. lactis Bi-07 (Accession no. NC_017867), Bifidobacterium animalis subsp. lactis B420 (Accession no. NC_017866), Bifidobacterium animalis subsp. lactis BB-12 (Accession no. CP001853), Bifidobacterium animalis subsp. lactis AD011 (Accession no. CP001213), Bifidobacterium animalis subsp. lactis HN019 (Accession no. CP031154), Bifidobacterium animalis subsp. lactis DN-173 010, Bifidobacterium animalis subsp. lactis ATCC 27536, or Bifidobacterium animalis subsp. lactis VTT E-012010.

Bifidobacterium animalis subsp. lactis CNCM I-3446, also named NCC 2818, was deposited with the Collection Nationale de Cultures de Microorganismes (CNCM), Institut Pasteur, 25 rue du Docteur Roux, F-75724 PARIS Cedex 15, France, on 7th June 2005 and given the deposit number 1-3446.

In some embodiments, the Bifidobacterium lactis is a Bifidobacterium animalis subsp. lactis having at least 99% sequence identity to Bifidobacterium animalis subsp. lactis CNCM I-3446. In some embodiments, the Bifidobacterium lactis is a Bifidobacterium animalis subsp. lactis having at least 99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446.

In some embodiments, the Bifidobacterium lactis is Bifidobacterium animalis subsp. lactis CNCM I-3446.

### Supernatant Preparation

The Bifidobacterium lactis supernatant may be prepared by any suitable method. For example, preparation of bacterial supernatants is described in Moradi, M., et al., 2021. Enzyme and Microbial Technology, 143, p. 109722.

A Bifidobacterium lactis supernatant may be obtained or obtainable by a method comprising: (a) culturing a Bifidobacterium lactis in a culture media to provide a Bifidobacterium lactis fermentate; and (b) removing substantially all the Bifidobacterium lactis cells from the Bifidobacterium lactis fermentate.

Suitable culture conditions and processing steps will be well known to the skilled person. Exemplary culture conditions and processing steps are described herein.

### Culture Media

The Bifidobacterium lactis supernatant may be obtained or obtainable by culturing Bifidobacterium lactis in a culture media.

The Bifidobacterium lactis may be cultured in any suitable culture media. Suitable culture media will be well known to the skilled person. For example, Marsaux, B., et al., 2020. Nutrients, 12(8), p. 2268 describe a suitable culture media for Bifidobacterium animalis subsp. lactis CNCM I-3446 made of dextrose at 2.8%, yeast-derived amino acids at 3%, and vitamin C.

Suitably, the culture media may be a commercially available media, such as MRS broth. MRS broth is non-selective medium for profuse growth of lactic acid bacteria and may comprise about 2% glucose, about 0.4% yeast extract, and about 0.1% polysorbate and may have a pH of about 6.2.

The culture media may comprise sugar, yeast extract, vitamin C, and/or polysorbate. Suitably, the culture media may comprise sugar and yeast extract. Suitably, the culture media may comprise sugar, yeast extract, and vitamin C. Suitably, the culture media may comprise sugar, yeast extract, vitamin C, and polysorbate.

Suitable sugars will be well known to the skilled person and include glucose, dextrose and/or glucose syrup. Suitably, the culture media may comprise from about 1 wt % to about 6 wt %, or from about 2 wt % to about 4 wt % sugar.

Yeast extract is the water-soluble portion of autolyzed yeast. Suitably, the culture media may comprise from about 1 wt % to about 10 wt %, from about 1 wt % to about 6 wt %, or from about 2 wt % to about 4 wt % yeast extract.

Suitable sources of vitamin C (also known as ascorbate or ascorbic acid) will be well known to the skilled person. For example, sodium ascorbate is one of a number of mineral salts of ascorbic acid. Suitably, the culture media may comprise from about 0 wt % to about 0.5 wt %, or from about 0.1 wt % to about 0.2 wt % sodium ascorbate.

Suitable polysorbates will be well known to the skilled person and include, for example, polysorbate 80. Suitably, the culture media may comprise from about 0 wt % to about 1 wt %, or from about 0 wt % to about 0.3 wt % polysorbate.

Suitably, the culture may comprise any other suitable components such as minerals (e.g. manganese sulphate) and/or peptone (e.g. yeast peptone).

In some embodiments, the culture media comprises from about 1 wt % to about 6 wt % sugar, from about 1 wt % to about 6 wt % yeast extract, from about 0 wt % to about 0.5 wt % sodium ascorbate, and from about 0 wt % to about 1 wt % polysorbate.

In some embodiments, the culture media comprises from about 2 wt % to about 4 wt % sugar, from about 2 wt % to about 4 wt % yeast extract, from about 0.1 wt % to about 0.2 wt % sodium ascorbate, and from about 0 wt % to about 0.3 wt % polysorbate.

Suitable pH will be well known to the skilled person and may be adjusted by any suitable means. Suitably, the culture media may have a pH of below about 7, such as from about 5.5 to about 6.5, or about 6.

### Culture Conditions

The Bifidobacterium lactis may be cultured under any suitable conditions. Suitable culture conditions will be well known to the skilled person. For example, Marsaux, B., et al., 2020. Nutrients, 12(8), p. 2268 describe that Bifidobacterium animalis subsp. lactis CNCM I-3446 was incubated at 37° C.

Suitably, the Bifidobacterium lactis supernatant may be obtained or obtainable by culturing the Bifidobacterium lactis under anaerobic conditions.

Suitably, the Bifidobacterium lactis supernatant may be obtained or obtainable by culturing the Bifidobacterium lactis until the late log phase or stationary phase is reached. In some embodiments, the Bifidobacterium lactis supernatant is obtained or obtainable by culturing the Bifidobacterium lactis until the stationary phase is reached.

The stages recognised in cultivation of bacteria are known to the skilled person and include the “lag phase”, the “log phase”, the “stationary phase”, and the “death” phase. The “lag phase” is the phase in which there is no increase in the number of living bacterial cells. The “log phase” is the phase in which there is an exponential increase in the number of living bacterial cells. The “late log phase” may refer to the second half of the log phase, for example at least about 75%, at least about 80%, at least about 85%, at least about 90%, or at least about 95% through the log phase. The “stationary phase” is the phase in which the number of living bacterial cells remains constant. The “death phase” is the phase in which there is a decline in the number of living bacterial cells.

The culture phase may be determined using any suitable method known to the skilled person. For example, the beginning of the stationary phase may be determined as the time point when no additional acids are produced and base addition is no longer required to maintain the desired pH. Alternatively, the culture phase may be determined based on the optical density at 600 nm, which correlates with the bacterial concentration in a culture medium.

Suitably, the Bifidobacterium lactis supernatant may be obtained or obtainable by culturing at about 35° C. to about 40° C., or about 37° C. The temperature may be controlled by any suitable method known to the skilled person.

Suitably, the Bifidobacterium lactis supernatant may be obtained or obtainable by culturing with pH control. The pH may be controlled by any suitable method known to the skilled person, for example by using base addition. Suitably, the pH may be controlled at a pH of from about 5 to about 7, from about 5.5 to about 6.5, or about 6. Suitably, the pH may be controlled at a pH of 6.0.

Suitably, the Bifidobacterium lactis supernatant may be obtained or obtainable by culturing with stirring and/or with head space gassing. The stirring and/or head space gassing may be carried out by any suitable method known to the skilled person, for example by head space gassing with carbon dioxide.

Suitably, the Bifidobacterium lactis supernatant may be obtained or obtainable by culturing at about 37° C., with pH control at about pH 6, with stirring, and/or with head space gassing with carbon dioxide.

### Processing of Fermentate

The Bifidobacterium lactis supernatant may be obtained or obtainable by removing all or substantially all the Bifidobacterium lactis cells from a Bifidobacterium lactis fermentate.

The Bifidobacterium lactis cells may be removed by any suitable method known to the skilled person. For example, the Bifidobacterium lactis cells may be removed by centrifugation or filtration.

The Bifidobacterium lactis cells may be removed by centrifugation. Suitable centrifugation conditions will be well known to the skilled person, for example, centrifugation at from about 4000 to about 12000 g for about 10 min.

The Bifidobacterium lactis cells may be removed by filtration. Suitable filtration conditions will be well known to the skilled person, for example membrane filtration using a pore size of about 0.2 μm to remove all the Bifidobacterium lactis cells or a pore size of from about 0.3 μm to about 0.5 μm to remove substantially all the Bifidobacterium lactis cells, or a 0.5 μm to 2.0 μm pore size filter may be used to remove substantially all Bifidobacterium lactis cells.

As used herein, “substantially all” may mean the vast majority of Bifidobacterium lactis cells, e.g. at least 90%, at least 95%, or at least 99% of the Bifidobacterium lactis cells. Suitably, removing “substantially all” the Bifidobacterium lactis cells may mean that at least about 90%, at least about 95%, or at least about 99% of the Bifidobacterium lactis cells are removed.

In some embodiments, some Bifidobacterium lactis cells remain in the supernatant (e.g. following removal). For example, prior to inactivation, the Bifidobacterium lactis supernatant may have a viable cell count of from about 1×10⁶ to about 1×10¹⁰ cfu/ml or a viable cell count of from about 1×10⁷ to about 1×10⁹ cfu/ml.

In some embodiments, the remaining Bifidobacterium lactis cells are heat-inactivated. Suitable methods to heat-inactivate the Bifidobacterium lactis cells will be well known to the skilled person. For example, the Bifidobacterium lactis supernatant may be pasteurised. Suitable pasteurisation conditions will be well known to the skilled person. For example, the Bifidobacterium lactis supernatant may be pasteurised at a temperature of from about 70° C. to about 100° C. for about 10 to about 30 seconds, about 70° C. to about 100° C. for about 10 to about 15 seconds, or about 80° C. to about 100° C. for about 10 seconds.

In some other embodiments, the Bifidobacterium lactis supernatant may be a cell-free supernatant (i.e. 100% of the Bifidobacterium lactis cells are removed). A cell-free supernatant may be obtained by passing the supernatant through one or more filters to remove all the Bifidobacterium lactis cells. Suitable filtering conditions will be well known to the skilled person.

For example, a 0.22 μm or a 0.4 μm pore size filter may be used to remove all Bifidobacterium lactis cells.

### Drying of Supernatant

Suitably, the Bifidobacterium lactis supernatant may be dried. Providing the Bifidobacterium lactis supernatant in a dry form may be more suitable for long-term storage. The Bifidobacterium lactis supernatant may be dried by any suitable method known to the skilled person. For example, the supernatant may be dried by spray-drying or lyophilisation. For the avoidance of doubt, the term “supernatant” includes a dried supernatant, e.g. a supernatant in a solid (e.g. powder) form).

In some embodiments, the Bifidobacterium lactis supernatant is spray-dried. Suitable methods for spray-drying a Bifidobacterium lactis supernatant will be well known to the skilled person (see e.g. Santos, D., et al., 2018. Biomaterials Physics and Chemistry—New Edition. InTech Open, Spray Drying: An Overview). For the avoidance of doubt, the term “supernatant” includes a spray-dried supernatant.

Suitably, the Bifidobacterium lactis supernatant may be spray-dried with a carrier material. Suitable carrier materials will be well known to the skilled person. For example, the carrier material may be selected from one or more of: oat fibre, maltodextrin, acacia gum, starch and inulin. In some embodiments, the carrier material is acacia gum.

Any suitable amount of carrier material may be used. For example, the Bifidobacterium lactis supernatant and carrier material may be mixed in a total solids ratio of from about 1:5 to about 5:1 (carrier:supernatant dry solids), or a total solids ratio of from about 1:3 to about 3:1 (carrier:supernatant dry solids), or a total solids ratio of from about 1:2 to about 2:1 (carrier:supernatant dry solids), or a total solids ratio of from about 1:1.5 to about 1.5:1 (carrier:supernatant dry solids), or a total solids ratio of from about 1:1 (carrier:supernatant dry solids).

### Form of Supernatant

The Bifidobacterium lactis supernatant may be provided in any suitable form. For example, the Bifidobacterium lactis supernatant can be in a solid (e.g. powder), liquid or gelatinous form. The Bifidobacterium lactis supernatant may be provided in a form suitable for oral or enteral administration. In some embodiments, the Bifidobacterium lactis supernatant is provided in a form suitable for oral administration.

In some embodiments, the Bifidobacterium lactis supernatant is provided in a solid (e.g. powder) form. Providing the Bifidobacterium lactis supernatant in the form of a solid (e.g. a powder) may be more suitable for consumption as a supplement (e.g. in tablet or capsule form).

The Bifidobacterium lactis supernatant may be provided in the form of a supplement or a nutritional composition.

In one aspect, the present invention provides a supplement comprising a Bifidobacterium lactis supernatant.

A “supplement” or “dietary supplement” may be used to complement the nutrition of an individual (it is typically used as such but it might also be added to any kind of compositions intended to be ingested). The supplement may be prepared in any suitable manner.

The supplement may be in the form of for example tablets, capsules, pastilles or a liquid. In some embodiments, the supplement is in the form of a capsule or a tablet.

The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the supplement may contain an organic or inorganic carrier material suitable for oral or parenteral administration as well as vitamins, minerals trace elements and other micronutrients in accordance with the recommendations of Government bodies such as the USRDA. The supplement may be provided in the form of unit doses.

In some embodiments, the supplement is a pet supplement. A “pet supplement” may refer to a supplement that is intended for pets. A pet may be an animal selected from dogs, cats, birds, fish, rodents such as mice, rats, and guinea pigs, rabbits, etc.

In one aspect, the present invention provides a nutritional composition comprising a Bifidobacterium lactis supernatant.

According to the present invention, a “nutritional composition” means a composition which nourishes a subject. The nutritional composition may be prepared in any suitable manner.

The nutritional composition is not particularly limited as long as it is suitable for administration (e.g. oral or intravenous administration). Examples of suitable nutritional compositions include foodstuffs, drinks, drug bases, and animal feeds.

The nutritional composition according to the invention may be an enteral nutritional composition. An “enteral nutritional composition” is a foodstuff that involves the gastrointestinal tract for its administration.

The nutritional composition may be suitable for infants. For example the nutritional composition may be an infant formula, a baby food, an infant cereal composition, or a fortifier. Suitably, the nutritional composition may be an infant formula or a fortifier.

Suitably, the nutritional composition may be a pharmaceutical composition. The form of the pharmaceutical preparation is not particularly limited, and examples include tablet, pill, powder, solution, suspension, emulsion, granule, capsule, syrup, and so forth. Additives widely used as pharmaceutical carriers for oral administration such as excipients, binders, disintegrating agents, lubricants, stabilizers, corrigents, diluents, and surfactants can be used. Examples of suitable binders include starch, gelatin, natural sugars such as glucose, anhydrous lactose, free-flow lactose, beta-lactose, corn sweeteners, natural and gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like.

In some embodiments, the nutritional composition is an animal feed, for example a pet food product (particularly a dry pet food product). A “pet food product” may refer to a nutritional product that is intended for consumption by pets. In some embodiments, the nutritional composition is a dog food product or a cat food product. In some embodiments, the nutritional composition is a veterinary composition.

### Supernatant Composition

As described above, the Bifidobacterium lactis supernatant may be obtained or obtainable by a method comprising (a) culturing a Bifidobacterium lactis in a culture media. During culturing the Bifidobacterium lactis will consume nutrients in the media (e.g. sugar and amino acids) and enrich the media with various soluble factors (e.g. organic acids and other metabolites).

The Bifidobacterium lactis-derived metabolites in a Bifidobacterium lactis supernatant may have diverse physicochemical and functional properties and can be analysed by any suitable method known to those of skill in the art. For example, suitable methods include gas chromatography, liquid chromatography, thin layer chromatography, spectrophotometric techniques, NMR spectroscopy, and FTIR spectroscopy (see e.g. Moradi, M., et al., 2021. Enzyme and Microbial Technology, 143, p. 109722).

Compared to Bifidobacterium lactis culture media the Bifidobacterium lactis fermentate or Bifidobacterium lactis supernatant may have: (i) a decreased concentration of total sugar; (ii) an increased concentration of total acids; and/or (iii) a decreased concentration of total amino acids.

As used herein, the term “total sugars” may refer to any sugar, including e.g. glucose and fructose. Suitably, compared to Bifidobacterium lactis culture media the concentration of total sugar in the Bifidobacterium lactis fermentate or Bifidobacterium lactis supernatant (e.g. prior to drying) may have decreased by at least about 50%, at least about 60%, at least about 70%, at least about 80%, at least about 90%, at least about 95%, or at least about 99%. Suitably, compared to Bifidobacterium lactis culture media the concentration of total sugar in the Bifidobacterium lactis fermentate or Bifidobacterium lactis supernatant (e.g. prior to drying) may have decreased by from about 50% to about 100%, or from about 80% to about 100%.

As used herein, the term “total acids” may refer to any organic acid (i.e. excluding amino acids), including e.g. acetic acid, lactic acid, and formic acid. Suitably, compared to Bifidobacterium lactis culture media the concentration of total acids in the Bifidobacterium lactis fermentate or Bifidobacterium lactis supernatant (e.g. prior to drying) may have increased by at least about 70%, by at least about 80%, or by at least about 90% of the concentration decrease in total sugars. For example, if the concentration of total sugars has decreased by 1 wt %, the concentration of total acids may have increased by at least about 0.7 wt %, at least about 0.8 wt %, or at least about 0.9 wt %. Suitably, compared to Bifidobacterium lactis culture media the concentration of total acids in the Bifidobacterium lactis fermentate or Bifidobacterium lactis supernatant (e.g. prior to drying) may have increased by from about 70% to about 90% of the concentration decrease in total sugars.

Suitably, compared to Bifidobacterium lactis culture media the concentration of total amino acids in the Bifidobacterium lactis fermentate or Bifidobacterium lactis supernatant (e.g. prior to drying) may have decreased by at least about 0.1 wt %, by at least about 0.2 wt %, by at least about 0.3 wt %, by at least 0.4 wt %, or by at least 0.5 wt %. Suitably, compared to Bifidobacterium lactis culture media the concentration of total amino acids in the Bifidobacterium lactis fermentate or Bifidobacterium lactis supernatant (e.g. prior to drying) may have decreased by from about 0.1 wt % to about 0.5 wt %.

Suitably, the Bifidobacterium lactis fermentate or Bifidobacterium lactis supernatant (e.g. prior to drying) may have a total solids content of from about 2 wt % to about 18 wt %, from about 2 wt % to about 10 wt %, from about 3 wt % to about 9 wt %, from about 4 wt % to about 8 wt %, from about 5 wt % to about 7 wt %, or about 6 wt %.

Suitably, the Bifidobacterium lactis fermentate or Bifidobacterium lactis supernatant (e.g. prior to drying) may comprise about 5 wt % or less, about 4 wt % or less, about 3 wt % or less, about 2 wt % or less, about 1 wt % or less, about 0.5 wt % or less, about 0.4 wt % or less, about 0.3 wt % or less, about 0.2 wt % or less, or about 0.1 wt % or less total sugars. In some embodiments, the supernatant may comprise less than 0.3 wt %, less than 0.2 wt %, or less than 0.1 wt % total sugars. Suitably, the Bifidobacterium lactis fermentate or Bifidobacterium lactis supernatant (e.g. prior to drying) may comprise about 0 wt % to about 5 wt %, about 0 wt % to about 2 wt %, about 0 wt % to about 1 wt %, about 0 wt % to about 0.5 wt %, or about 0 wt % to about 0.3 wt % total sugars.

Suitably, the Bifidobacterium lactis fermentate or Bifidobacterium lactis supernatant (e.g. prior to drying) may comprise about 0.5 wt % or more, about 1 wt % or more, about 1.5 wt % or more, about 2 wt % or more, about 3 wt % or more, about 4 wt % or more, or about 5 wt % or more total acids. Suitably, the Bifidobacterium lactis fermentate or Bifidobacterium lactis supernatant (e.g. prior to drying) may comprise about 0.5 wt % to about 5 wt %, about 1 wt % to about 3 wt %, about 1.5 wt % to about 2.5 wt %, about 2 wt % to about 2.5 wt %, or about 2 wt % total acids.

Suitably, the Bifidobacterium lactis fermentate or Bifidobacterium lactis supernatant (e.g. prior to drying) may comprise about 0.5 wt % to about 6 wt %, about 1 wt % to about 3 wt %, or about 1.5 wt % to about 2.5 wt % ash.

Suitably, the Bifidobacterium lactis fermentate or Bifidobacterium lactis supernatant (e.g. prior to drying) may comprise about 3.5 wt % or less, about 3 wt % or less, about 2.5 wt % or less, about 2 wt % or less, about 1.5 wt % or less, about 1 wt % or less, about 0.8 wt % or less, about 0.6 wt % or less, about 0.4 wt % or less, or about 0.2 wt % or less total amino acids. Suitably, the Bifidobacterium lactis fermentate or Bifidobacterium lactis supernatant (e.g. prior to drying) may comprise about 0.2 wt % to about 3.5 wt %, about 0.4 wt % to about 2 wt %, about 0.6 wt % to about 1 wt %, about 0.7 wt % to about 0.9 wt %, or about 0.8 wt % total amino acids.

Suitably, the Bifidobacterium lactis fermentate or Bifidobacterium lactis supernatant (e.g. prior to drying) may comprise: about 5 wt % or less total sugars; about 0.5 wt % or more total acids; and about 3.5 wt % or less total amino acids.

Suitably, the Bifidobacterium lactis fermentate or Bifidobacterium lactis supernatant (e.g. prior to drying) may comprise: about 2 wt % or less total sugars; about 1 wt % or more total acids; and about 2 wt % or less total amino acids.

Suitably, the Bifidobacterium lactis fermentate or Bifidobacterium lactis supernatant (e.g. prior to drying) may comprise: about 1 wt % or less total sugars; about 1.5 wt % or more total acids; and about 1 wt % or less total amino acids.

Suitably, the Bifidobacterium lactis fermentate or Bifidobacterium lactis supernatant (e.g. prior to drying) may comprise: about 0.5 wt % or less total sugars; about 2 wt % or more total acids; and about 0.8 wt % or less total amino acids.

Suitably, the Bifidobacterium lactis fermentate or Bifidobacterium lactis supernatant (e.g. prior to drying) may comprise: about 0 wt % to about 5 wt % total sugars; about 0.5 wt % to about 5 wt % total acids; and about 0.2 wt % to about 3.5 wt % or less total amino acids.

Suitably, the Bifidobacterium lactis fermentate or Bifidobacterium lactis supernatant (e.g. prior to drying) may comprise: about 0 wt % to about 2 wt % total sugars; about 1 wt % to about 3 wt % total acids; and about 0.4 wt % to about 2 wt % total amino acids.

Suitably, the Bifidobacterium lactis fermentate or Bifidobacterium lactis supernatant (e.g. prior to drying) may comprise: about 0 wt % to about 1 wt % total sugars; about 1.5 wt % to about 2.5 wt % total acids; and about 0.6 wt % to about 1 wt % total amino acids.

Suitably, the Bifidobacterium lactis fermentate or Bifidobacterium lactis supernatant (e.g. prior to drying) may comprise: about 0 wt % to about 0.3 wt % total sugars; about 2 wt % total acids; and about 0.8 wt % total amino acids.

Suitably, the Bifidobacterium lactis fermentate or Bifidobacterium lactis supernatant (e.g. prior to drying) may comprise:
(i) less than about 0.3 wt % glucose;
(ii) less than about 0.3 wt % fructose;
(iii) about 0 wt % to about 0.5 wt % citric acid;
(iv) about 0.6 wt % to about 0.9 wt % lactic acid;
(v) about 0.1 wt % to about 0.3 wt % formic acid;
(vi) about 1.2 wt % to about 1.4 wt % acetic acid; and/or
(vii) about 0.2 wt % total nitrogen.

Suitably, the Bifidobacterium lactis fermentate or Bifidobacterium lactis supernatant (e.g. prior to drying) may comprise: about 0 wt % to about 2 wt % total sugars, including less than about 0.3 wt % glucose and less than about 0.3 wt % fructose; about 1.5 wt % to about 2.5 wt % total acids, including about 0 wt % to about 0.5 wt % citric acid, about 0.6 wt % to about 0.9 wt % lactic acid, about 0.1 wt % to about 0.3 wt % formic acid, and about 1.2 wt % to about 1.4 wt % acetic acid; and about 0.6 wt % to about 1 wt % total amino acids, including about 0.2 wt % total nitrogen.

### Probiotic, Prebiotic, and Synbiotic Combinations

The Bifidobacterium lactis supernatant may be used in combination with one or more probiotics, prebiotics, or synbiotics. In some embodiments, the Bifidobacterium lactis supernatant is used in combination with one or more probiotics. The probiotics, prebiotics, or synbiotics and Bifidobacterium lactis supernatant may be combined in any suitable doses.

In one aspect, the present invention provides a supplement or nutritional composition comprising a combination of a Bifidobacterium lactis supernatant and one or more probiotics, prebiotics, or synbiotics.

In some embodiments, the present invention provides a supplement or nutritional composition comprising a combination of a Bifidobacterium lactis supernatant and one or more probiotics.

The term “probiotic” may refer to a component that contains live microorganisms that, when administered in adequate amounts, confer a health benefit on the subject (see e.g. Hill, C., et al., 2014. Nature reviews Gastroenterology & hepatology, 11(8), p. 506).

Suitably, the probiotic may comprise a commercially available probiotic strain and/or a strain which has been shown to have health benefits (See e.g. Fijan, S., 2014. International journal of environmental research and public health, 11(5), pp. 4745-4767). In some embodiments, the probiotic comprises Escherichia, Bifidobacterium, Streptococcus, Lactobacillus (as defined up to March 2020), Bacillus, and/or Enterococcus.

In some embodiments, the probiotic is a Bifidobacterium lactis probiotic. Any suitable Bifidobacterium lactis strain may be used, for example any Bifidobacterium lactis strain which is known to have a probiotic effect. Such Bifidobacterium lactis strains will be well known to the skilled person and are described above in the section entitled “Bifidobacterium lactis strains”. The Bifidobacterium lactis may be the same Bifidobacterium lactis from which the supernatant is derived or may be different. In some embodiments, the Bifidobacterium lactis is the same Bifidobacterium lactis from which the supernatant is derived.

The term “prebiotic” may refer to a non-digestible component that benefits the subject by selectively stimulating the favourable growth and/or activity of one or more microbial taxa. Exemplary prebiotics include human milk oligosaccharides. Exemplary prebiotic oligosaccharides include galacto-oligosaccharides (GOS), fructo-oligosaccharides (FOS), 2′-fucosyllactose, lacto-N-neo-tetraose, and inulin.

The term “synbiotic” may refer to a component that contains both probiotics and prebiotics (see e.g. Swanson, K. S., et al., 2020. Nature Reviews Gastroenterology & Hepatology, 17(11), pp. 687-701).

### Use as an Immunomodulator

As described above, Bifidobacterium lactis supernatant has surprisingly been found to exhibit immune-modulatory effects superior in several aspects to those of the B. lactis probiotic, which is well-documented in the literature for its immune-modulatory effects in various clinical trials.

In one aspect, the present invention provides use of a Bifidobacterium lactis supernatant as an immunomodulator. As used herein, an “immunomodulator” is a substance that affects the functioning of the immune system. In some embodiments, the use of a Bifidobacterium lactis supernatant as an immunomodulator enhances expression of anti-inflammatory cytokines (e.g. IL-10) and/or reduces expression of pro-inflammatory chemokines in the gastrointestinal tract of a subject.

In another aspect, the present invention provides use of a Bifidobacterium lactis supernatant as an immunosuppressant. As used herein, an “immunosuppressant” is a substance that inhibit or prevent activity of the immune system. In some embodiments, the use of a Bifidobacterium lactis supernatant as an immunosuppressant enhances expression of anti-inflammatory cytokines (e.g. IL-10) and/or reduces expression of pro-inflammatory chemokines in the gastrointestinal tract of a subject.

In another aspect, the present invention provides a Bifidobacterium lactis supernatant for use in enhancing expression of anti-inflammatory cytokines (e.g. IL-10) and/or reducing expression of pro-inflammatory chemokines in the gastrointestinal tract of a subject.

The Bifidobacterium lactis supernatant may enhance the expression of one or more anti-inflammatory cytokines in the gastrointestinal tract of a subject. The anti-inflammatory cytokines are a series of immunoregulatory molecules that control the pro-inflammatory cytokine response (see e.g. Opal, S. M. and DePalo, V. A., 2000. Chest, 117(4), pp. 1162-1172). For, example the Bifidobacterium lactis supernatant may enhance the expression of one or more anti-inflammatory cytokine selected from: IL-1 receptor antagonist, IL-4, IL-6, IL-10, IL-11, and IL-13. In some embodiments, the Bifidobacterium lactis supernatant enhances the expression of IL-6 and/or IL-10. In some embodiments, the Bifidobacterium lactis supernatant enhances the expression of IL-10.

The Bifidobacterium lactis supernatant may reduce the expression of one or more pro-inflammatory chemokines in the gastrointestinal tract of a subject. Pro-inflammatory chemokines are those upregulated under inflammatory conditions and are mainly involved in the recruitment of leukocytes to inflamed tissues (see e.g. Zlotnik, A. and Yoshie, O., 2012. Immunity, 36(5), pp. 705-716). For, example the Bifidobacterium lactis supernatant may reduce the expression of one or more pro-inflammatory chemokines selected from: CXCL-8 (IL-8), CCL2, CCL3, CCL4, CCL5, CCL11, CXCL10. In some embodiments, the Bifidobacterium lactis supernatant reduces the expression of CXCL10 and/or IL-8. In some embodiments, the Bifidobacterium lactis supernatant reduces the expression of IL-8.

### Interleukin-10 (IL-10)

In one aspect, the present invention provides a Bifidobacterium lactis supernatant for use in enhancing expression of IL-10 in the gastrointestinal tract of a subject.

Interleukin-10 (IL-10) is a pleiotropic, immunoregulatory cytokine that is important in protecting the host from allergy, infection-associated immunopathology and autoimmunity. IL-10 was initially characterized as a T helper (TH) 2 specific cytokine; however, further investigations revealed that IL-10 production was also associated with T regulatory (Treg) cell responses. IL-10-deficient mice exhibit prolonged and exaggerated immune responses toward antigen, in many cases accompanied by excessive inflammation and tissue damage, and they often develop chronic enterocolitis (Kühn et al., 1993, Cell 75, 263-274; Leon et al., 1998, Ann. N. Y. Acad. Sci. 856, 69-75.). Single-nucleotide polymorphisms (SNPs) associated with lower IL-10 mRNA expression are also overrepresented in patients with rheumatoid arthritis (RA) (Hajeer et al., 1998, Scand. J. Rheumatol. 27, 142-145), severe asthma (Lim et al., 1998, Lancet 352, 113,), and systemic lupus erythematosus (SLE) (Gibson et al., 2001, J. Immunol. 166, 3915-3922). Thymically and Peripherally Generated FoxP3+ Regulatory T Cells Secrete IL-10 (Sky Ng et al., Front. Immunol., 31 May 2013).

The cytokines IL-4, IL-5, and IL-13, secreted by TH2 cells, provide protective immunity in the context of parasite infection, but also initiate, amplify, and prolong allergic responses by enhancing production of IgE and are responsible for recruitment, expansion, and differentiation of eosinophils and mast cells (Robinson et al., 1992, N. Engl. J. Med. 326, 298-304; Romagnani, 1994, Annu. Rev. Immunol. 12, 227-257; Northrop et al., 2006, J. Immunol. 177, 1062-1069). Early studies of experimental TH2-inducing parasitic infections, including Trichuris muris and T. cruzii demonstrated a key role for IL-10 in preventing a lethal T cell response (Schopf et al., 2002, J. Immunol. 168, 2383-2392).

TH2-derived IL-10 is associated with downregulation of IL-4 and IL-13 during allergic responses (Grünig et al., 1997, J. Exp. Med. 185, 1089-1100; Jutel et al., 2003, Eur. J. Immunol. 33, 1205-1214; Akdis et al., 2004, J. Exp. Med. 199, 1567-1575). In a mouse model of allergic airway inflammation, IL-10 is crucial in restraining TH2 responses (Grünig et al., 1997). After repeated inhalation of Aspergillus fumigatus allergens, lung cells and broncho-alveolar lavage (BAL) fluid from IL-10-knockout mice produced higher levels of IL-4, IL-5, and IFN-γ, leading to exaggerated airway inflammation (Grünig et al., 1997). In addition, alveolar macrophages isolated from asthmatic patients secrete lower levels of IL-10 compared to those from non-asthmatics (Borish, 1998; John et al., 1998, Am. J. Respir. Crit. Care Med. 157, 256-262).

IL-10 plays an important role in mediating successful antigen-specific therapeutic tolerance. For example, intranasal administration of peptide derived from ovalbumin (OVA) can reduce symptoms of TH2-driven OVA/alum-induced airway hypersensitivity (AHR) (Akbari et al., 2001). Protection from AHR is associated with induction of IL-10-secreting pulmonary DCs with capacity to induce IL-4 and IL-10-secreting OVA-specific CD4+ T cells in vitro (Akbari et al., 2001). Neutralization of IL-10 during tolerance induction results in elevated OVA-specific IgE production and negates the protective effect of OVA administration (Vissers et al., 2004, J. Allergy Clin. Immunol. 113, 1204-1210). Successful allergen-specific immunotherapy (SIT) in man, for example in the treatment of grass pollen or house dust mite allergies, correlates with generation of IL-10-secreting CD4+ T cells (Jutel et al., 2003, Eur. J. Immunol. 33, 1205-1214). IL-10 limits TH2 responses by downregulation of IL-4, inhibition of antigen presentation by MHC class II on DCs, and suppression of co-stimulatory molecule expression including CD28, ICOS, and CD2 (Taylor et al., 2007, J. Allergy Clin. Immunol. 120, 76-83). This is mediated via src homology phosphatase (SHP)-1 in naïve CD4+ T cells, suggesting that IL-10 can regulate effector responses and also prevent the differentiation of TH2 cells from naîve CD4+ T cells (Taylor et al., 2007).

The critical role of IL-10 in immunoregulation goes beyond the prevention of allergic disease and extends to other diseases including inflammatory bowel disease and autoimmune disease. In the context of IBD it has been showed that mice deficient in IL-10 develop spontaneous colitis (Kuhn et al; 1993, Cell. 75, 263-274). This process can be prevented by IL-10 administration or IL-10 overexpression (Steidler et al; 2000, Science. 289, 1352-1355 and Hagenbaugh et al; 1997, J Exp Med. 185, 2101-2110). Similarly, IBD predisposition in humans is strongly associated with defect IL-10 responses (Glocker et al; 2009, N Engl J Med. 361, 2033-2045). The importance of IL-10 in immunoregulation has further been demonstrated in a range of autoimmune pathologies as lack of IL-10 worsened the development of rheumatoid arthritis (Hata et al; 2004, J Clin Invest. 114, 582-588), lupus (Ishida et al; 1994, J Exp Med. 179, 305-310) and encephalomyelitis (Betteli et al; 1998, J Immunol. 161, 3299-3306) in preclinical models.

### Routes of Administration

The Bifidobacterium lactis supernatant, supplement or nutritional composition may be administered by any suitable method known to the skilled person. For example, the Bifidobacterium lactis supernatant, supplement or nutritional composition may be administered by oral and/or enteral administration. In some embodiments, the Bifidobacterium lactis supernatant, supplement or nutritional composition is orally administered.

### Subject

The Bifidobacterium lactis supernatant may be administered to a subject, wherein the subject is a mammal. Suitably, the subject is a human or a pet, such as a dog, a cat, a rodent (e.g. a mouse, a rat, or a guinea pig), or a rabbit. Preferably, the subject is a human subject.

The subject may be any age. For example, the subject may be a child or an adult. The term “child” may refer to a subject aged under 18 years. The term “adult” may refer to a subject aged 18 years or older. In some embodiments, the subject is a child. In some embodiments, the subject is an adult.

In some embodiments, the subject is an infant, a toddler, or a young child. The term “infant” may refer to a subject aged from about 0 years to about 1 year. The term “toddler” may refer to a subject aged from about 1 year to about 3 years. The term “young child” may refer to a subject aged from about 3 years to about 5 years. In some embodiments, the infant, toddler, or young child is a preterm infant, toddler, or young child. A “preterm” or “premature”, toddler, or young child means an infant, toddler, or young child who was not born at term (e.g. born prior 36 weeks of gestation). In some embodiments, the infant, toddler, or young child was born by C-section or was vaginally delivered.

The subject may have or may be at risk of an IL-10 deficiency. Whether a subject is IL-10 deficient may be determined by any method known to the skilled person. Affected patients primarily present early in life with inflammatory bowel disease (IBD). IL-10 mutations can be screened for by gene sequencing (see e.g. Glocker, E. O., et al., 2011. Annals of the New York Academy of Sciences, 1246(1), pp. 102-107).

The subject may have or may be at risk of an overactive immune system disorder. Such overactive immune system disorders are described in more detail in the section entitled “Methods of treating and/or preventing an overactive immune system disorder”. In some embodiments, the subject has or is at risk of an IL-10 mediated disease. In some embodiments, the subject has or is at risk of an inflammatory bowel disease. In some embodiments, the subject has or is at risk of an allergic disease. In some embodiments, the subject has or is at risk of dermatitis. In some embodiments, the subject has or is at risk of an autoimmune disease. In some embodiments, the subject has or is at risk of an infection-associated immunopathology. In some embodiments, the subject has or is at risk of colorectal cancer, impaired bone healing, or atherosclerosis.

The subject may have or may be at risk of an IL-6 deficiency. Whether a subject is IL-6 deficient may be determined by any method known to the skilled person.

### Methods of Treating or Preventing Disease

In one aspect, the present invention provides a Bifidobacterium lactis supernatant for use as a medicament. In another aspect, the present invention provides use of a Bifidobacterium lactis supernatant for the manufacture of a medicament. In another aspect, the present invention provides a method of treatment comprising administering a Bifidobacterium lactis supernatant.

In one aspect, the present invention provides a supplement or nutritional composition comprising a Bifidobacterium lactis supernatant for use as a medicament. In another aspect, the present invention provides use of a supplement or nutritional composition comprising a Bifidobacterium lactis supernatant for the manufacture of a medicament. In another aspect, the present invention provides a method of treatment comprising administering a supplement or nutritional composition comprising a Bifidobacterium lactis supernatant.

The Bifidobacterium lactis supernatant, supplement, or nutritional composition may prevent or treat a disease by enhancing expression of anti-inflammatory cytokines (e.g. IL-10) and/or reducing expression of pro-inflammatory chemokines in the gastrointestinal tract of the subject.

### Methods of Treating or Preventing an Overactive Immune System Disorder

Dysregulation of anti-inflammatory cytokines and/or pro-inflammatory chemokines can result in chronic systemic inflammation and an overactive immune system.

In one aspect, the invention provides a Bifidobacterium lactis supernatant for use in treating and/or preventing an overactive immune system disorder. In another aspect, the invention provides for use of a Bifidobacterium lactis supernatant for the manufacture of a medicament for treating and/or preventing an overactive immune system disorder. In another aspect, the invention provides a method of treating and/or preventing an overactive immune system disorder in a subject, comprising administering a Bifidobacterium lactis supernatant to the subject.

In one aspect, the invention provides a supplement or nutritional composition comprising a Bifidobacterium lactis supernatant for use in treating and/or preventing an overactive immune system disorder. In another aspect, the invention provides a supplement or nutritional composition comprising a Bifidobacterium lactis supernatant for the manufacture of a medicament for treating and/or preventing an overactive immune system disorder. In another aspect, the invention provides a method of treating and/or preventing an overactive immune system disorder in a subject, comprising administering a supplement or nutritional composition comprising a Bifidobacterium lactis supernatant to the subject.

As used herein, an “overactive immune system disorder” (also known as a “hyperactive immune system disorder”) may refer to a disease caused by chronic systemic inflammation and/or an overactive immune system. Overactive immune system disorders may include, for example allergic diseases and autoimmune diseases.

In some embodiments, the overactive immune system disorder is associated with an IL-10 deficiency. In some embodiments, the overactive immune system disorder is associated with an IL-6 deficiency. In some embodiments, the overactive immune system disorder is selected from: an inflammatory bowel disease (IBD), an allergic disease, dermatitis, an autoimmune disease, an infection-associated immunopathology, colorectal cancer, impaired bone healing, and atherosclerosis.

### IL-10 Mediated Diseases

An IL-10 deficiency can result in severe dysregulation of the immune system and can enhance inflammatory response to microbial challenge and lead to development of inflammatory bowel disease (IBD) and a number of autoimmune diseases (Iyer, S. S. and Cheng, G., 2012. Critical Review in Immunology, 32(1)).

In one aspect, the invention provides a Bifidobacterium lactis supernatant for use in treating and/or preventing an IL-10 mediated disease. In another aspect, the invention provides for use of a Bifidobacterium lactis supernatant for the manufacture of a medicament for treating and/or preventing an IL-10 mediated disease. In another aspect, the invention provides a method of treating and/or preventing an IL-10 mediated disease in a subject, comprising administering a Bifidobacterium lactis supernatant to the subject.

In one aspect, the invention provides a supplement or nutritional composition comprising a Bifidobacterium lactis supernatant for use in treating and/or preventing IL-10 mediated disease. In another aspect, the invention provides a supplement or nutritional composition comprising a Bifidobacterium lactis supernatant for the manufacture of a medicament for treating and/or preventing an IL-10 mediated disease. In another aspect, the invention provides a method of treating and/or preventing an IL-10 mediated disease in a subject, comprising administering a supplement or nutritional composition comprising a Bifidobacterium lactis supernatant to the subject.

### Inflammatory Bowel Diseases

Inflammatory bowel disease (IBD) is a group of inflammatory conditions of the colon and small intestine. Exemplary IBDs include Crohn's disease (CD), ulcerative colitis (UC), and pouchitis. It has been shown that serum IL-10 is increased during disease recovery in patients with inflammatory bowel disease (Mitsuyama, K., et al., 2006. Mediators of Inflammation, 2006(6), pp. 26875-26875).

IL-10 recombinant therapy has demonstrated efficacy in several preclinical models of IBD (Iyer, S. S. and Cheng, G., 2012. Critical Reviews in Immunology, 32(1), pp. 23-63; and Li, M. C. and He, S. H., 2004. World Journal of Gastroenterology, 10(5), p. 620).

In one aspect, the invention provides a Bifidobacterium lactis supernatant for use in treating and/or preventing an IBD. In another aspect, the invention provides for use of a Bifidobacterium lactis supernatant for the manufacture of a medicament for treating and/or preventing an IBD. In another aspect, the invention provides a method of treating and/or preventing an IBD in a subject, comprising administering a Bifidobacterium lactis supernatant to the subject.

In one aspect, the invention provides a supplement or nutritional composition comprising a Bifidobacterium lactis supernatant for use in treating and/or preventing an IBD. In another aspect, the invention provides a supplement or nutritional composition comprising a Bifidobacterium lactis supernatant for the manufacture of a medicament for treating and/or preventing an IBD. In another aspect, the invention provides a method of treating and/or preventing an IBD in a subject, comprising administering a supplement or nutritional composition comprising a Bifidobacterium lactis supernatant to the subject.

In some embodiments, the IBD is Crohn's disease (CD) or ulcerative colitis (UC).

### Allergic Diseases

Allergic diseases are a number of conditions caused by hypersensitivity of the immune system to typically harmless substances in the environment. Allergic diseases include hay fever, food allergies, atopic dermatitis, allergic asthma, and anaphylaxis.

Allergen-reactive T helper type-2 (Th2) cells and pro-inflammatory cytokines have been suggested to play an important role in the induction and maintenance of the inflammatory cascade in allergic asthma. Plasma IL-10 and IL-13 concentrations were significantly higher in allergic asthmatic patients than normal control subjects (Wong, C. K., et al., 2001. Clinical & Experimental Immunology, 125(2), pp. 177-183). Moreover, in animal models of asthma, IL-10 was shown to be capable of inhibiting allergen-induced airway inflammation and non specific responsiveness (Iyer, S. S. and Cheng, G., 2012. Critical Reviews in Immunology, 32(1), pp. 23-63).

Specific single nucleotide polymorphisms of the IL-10 gene seem to confer an increased risk of developing food allergy. Special interest has been drawn to the development of allergen-specific regulatory CD4+CD25+ T-cells secreting IL-10 in the immunotherapy of allergic diseases (Nedelkopoulou, N., et al., 2020. Allergologia et immunopathologia, 48(4), pp. 401-408).

In one aspect, the invention provides a Bifidobacterium lactis supernatant for use in treating and/or preventing an allergic disease. In another aspect, the invention provides for use of a Bifidobacterium lactis supernatant for the manufacture of a medicament for treating and/or preventing an allergic disease. In another aspect, the invention provides a method of treating and/or preventing an allergic disease in a subject, comprising administering a Bifidobacterium lactis supernatant to the subject.

In one aspect, the invention provides a supplement or nutritional composition comprising a Bifidobacterium lactis supernatant for use in treating and/or preventing an allergic disease. In another aspect, the invention provides a supplement or nutritional composition comprising a Bifidobacterium lactis supernatant for the manufacture of a medicament for treating and/or preventing an allergic disease. In another aspect, the invention provides a method of treating and/or preventing an allergic disease in a subject, comprising administering a supplement or nutritional composition comprising a Bifidobacterium lactis supernatant to the subject.

In some embodiments, the allergic disease is allergic asthma or a food allergy.

### Dermatitis

Dermatitis (also known as eczema) is inflammation of the skin, typically characterized by itchiness, redness and a rash. Dermatitis includes atopic dermatitis, allergic contact dermatitis, irritant contact dermatitis, seborrhoeic dermatitis and stasis dermatitis.

It has been shown that IL-10 may have a suppressive role in contact and atopic dermatitis (Boyman, O., et al., 2012. Journal of Allergy and Clinical Immunology, 129(1), pp. 160-161). For example, it has been shown that severe atopic dermatitis is associated with a reduced frequency of IL-10 producing allergen-specific CD4+ T cells (Seneviratne, S. L., et al., 2006. Clinical and Experimental Dermatology: Experimental dermatology, 31(5), pp. 689-694). Moreover, mast cell-derived IL-10 limits skin pathology in contact dermatitis (Grimbaldeston, M. A., et al., 2007. Nature immunology, 8(10), pp. 1095-1104).

In one aspect, the invention provides a Bifidobacterium lactis supernatant for use in treating and/or preventing dermatitis. In another aspect, the invention provides for use of a Bifidobacterium lactis supernatant for the manufacture of a medicament for treating and/or preventing dermatitis. In another aspect, the invention provides a method of treating and/or preventing dermatitis in a subject, comprising administering a Bifidobacterium lactis supernatant to the subject.

In one aspect, the invention provides a supplement or nutritional composition comprising a Bifidobacterium lactis supernatant for use in treating and/or preventing dermatitis. In another aspect, the invention provides a supplement or nutritional composition comprising a Bifidobacterium lactis supernatant for the manufacture of a medicament for treating and/or preventing dermatitis. In another aspect, the invention provides a method of treating and/or preventing dermatitis in a subject, comprising administering a supplement or nutritional composition comprising a Bifidobacterium lactis supernatant to the subject.

In some embodiments, the dermatitis is atopic dermatitis or contact dermatitis (e.g. allergic contact dermatitis).

### Autoimmune Diseases

An autoimmune disease is a condition arising from an abnormal immune response to a functioning body part. Autoimmune diseases are characterized by the presence of dysregulated cytokine expression that plays a role in the maintenance of autoreactive lymphocytes.

A number of studies of both mouse and human models of autoimmune diseases have documented altered IL-10 serum levels, suggesting a direct link between IL-10 levels and disease. IL-10 polymorphisms have been associated with autoimmune diseases including systemic lupus erythematosus, rheumatoid arthritis, multiple sclerosis, psoriasis, ankylosing spondylitis, Sjögren's syndrome, and Guillian-Barre syndrome (Gibson A W, et al. The Role of IL-10 in Autoimmune Pathology, In: Madame Curie Bioscience Database).

In one aspect, the invention provides a Bifidobacterium lactis supernatant for use in treating and/or preventing an autoimmune disease. In another aspect, the invention provides for use of a Bifidobacterium lactis supernatant for the manufacture of a medicament for treating and/or preventing an autoimmune disease. In another aspect, the invention provides a method of treating and/or preventing an autoimmune disease in a subject, comprising administering a Bifidobacterium lactis supernatant to the subject.

In one aspect, the invention provides a supplement or nutritional composition comprising a Bifidobacterium lactis supernatant for use in treating and/or preventing an autoimmune disease. In another aspect, the invention provides a supplement or nutritional composition comprising a Bifidobacterium lactis supernatant for the manufacture of a medicament for treating and/or preventing an autoimmune disease. In another aspect, the invention provides a method of treating and/or preventing an autoimmune disease in a subject, comprising administering a supplement or nutritional composition comprising a Bifidobacterium lactis supernatant to the subject.

In some embodiments, the autoimmune disease is selected from: systemic lupus erythematosus, rheumatoid arthritis, multiple sclerosis, psoriasis, ankylosing spondylitis, Sjögren's syndrome, and Guillian-Barre syndrome.

### Infection-Associated Immunopathology

For some infections the immune response can be the main cause of disease. Damage caused by the immune system is known as immunopathology and may be caused by antibodies, an excessive innate response, or lymphocytes.

IL-10 can both impede pathogen clearance and ameliorate immunopathology and has emerged as a key immunoregulator during infection with viruses, bacteria, fungi, protozoa, and helminths. Studies indicate that resolution of infection requires a coordinated response in which initial pro-inflammatory mechanisms clear the pathogen and are subsequently limited by IL-10 before pathology occurs. For example, ablation of IL-10 signaling results in the onset of severe, often fatal immunopathology in a number of infections including T. gondii, malaria, and Trypanosoma cruzi (Couper, K. N, et al., 2008. The Journal of Immunology, 180(9), pp. 5771-5777).

In one aspect, the invention provides a Bifidobacterium lactis supernatant for use in treating and/or preventing an infection-associated immunopathology. In another aspect, the invention provides for use of a Bifidobacterium lactis supernatant for the manufacture of a medicament for treating and/or preventing an infection-associated immunopathology. In another aspect, the invention provides a method of treating and/or preventing an infection-associated immunopathology in a subject, comprising administering a Bifidobacterium lactis supernatant to the subject.

In one aspect, the invention provides a supplement or nutritional composition comprising a Bifidobacterium lactis supernatant for use in treating and/or preventing an infection-associated immunopathology. In another aspect, the invention provides a supplement or nutritional composition comprising a Bifidobacterium lactis supernatant for the manufacture of a medicament for treating and/or preventing an infection-associated immunopathology. In another aspect, the invention provides a method of treating and/or preventing an infection-associated immunopathology in a subject, comprising administering a supplement or nutritional composition comprising a Bifidobacterium lactis supernatant to the subject.

In some embodiments, the infection is by a protozoa (e.g. Toxoplasma gondii, Leishmania spp., Plasmodium spp., Trypanosoma cruzi), a bacteria (e.g. Mycobacteria spp., Listeria monocytogenes, Helicobacter spp., Bordetella spp., Streptococcus pyogenes), a helminth (e.g. Schistosoma mansoni, Heligmosomoides polygyrus), a virus (e.g. HIV, hepatitis, HSV-1, LCMV, MCMV), or a fungus (e.g. Candida albicans).

### Other Diseases

Polymorphisms in IL-10 may be associated with colorectal cancer (Tsilidis, K. K, et al., 2009. Cancer Causes & Control, 20(9), pp. 1739-1751). Moreover, it has been shown that colorectal cancer is linked with an intensified production of a wide array of monocyte/macrophage pro-inflammatory cytokines which is not accompanied by elevated levels of circulating IL-10 (Szkaradkiewicz, A., et al., 2009. Archivum immunologiae et therapiae experimentalis, 57(4), pp. 291-294).

When a broken bone fails to heal it is called a “nonunion” and a “delayed union” is when a fracture takes longer than usual to heal. IL10 can affect bone formation by promoting chondrocyte proliferation and differentiation via the bone morphogenetic protein (BMP) pathway, thus influencing endochondral bone formation. IL-10 deficient mice showed suppressed bone formation and osteoblastogenesis, resulting in osteopenia and increased bone fragility (Maruyama, M., et al., 2020. Frontiers in Endocrinology, 11, p. 386).

Atherosclerosis, the formation of fibrofatty lesions in the artery wall, causes much morbidity and mortality worldwide, including most myocardial infarctions and many strokes, as well as disabling peripheral artery disease. It has been shown that IL-10 deficiency can play a deleterious role in atherosclerosis and that IL-10 may reduce atherogenesis and improve the stability of plaques (Caligiuri, G., et al., 2003. Molecular medicine, 9(1), pp. 10-17; and Mallat, Z., et al., 1999. Circulation research, 85(8), pp. e17-e24).

In one aspect, the invention provides a Bifidobacterium lactis supernatant for use in treating and/or preventing colorectal cancer, impaired bone healing, or atherosclerosis. In another aspect, the invention provides for use of a Bifidobacterium lactis supernatant for the manufacture of a medicament for treating and/or preventing colorectal cancer, impaired bone healing, or atherosclerosis. In another aspect, the invention provides a method of treating and/or preventing colorectal cancer, impaired bone healing, or atherosclerosis in a subject, comprising administering a Bifidobacterium lactis supernatant to the subject.

In one aspect, the invention provides a supplement or nutritional composition comprising a Bifidobacterium lactis supernatant for use in treating and/or preventing colorectal cancer, impaired bone healing, or atherosclerosis. In another aspect, the invention provides a supplement or nutritional composition comprising a Bifidobacterium lactis supernatant for the manufacture of a medicament for treating and/or preventing colorectal cancer, impaired bone healing, or atherosclerosis. In another aspect, the invention provides a method of treating and/or preventing colorectal cancer, impaired bone healing, or atherosclerosis in a subject, comprising administering a supplement or nutritional composition comprising a Bifidobacterium lactis supernatant to the subject.

### IL-6 Mediated Diseases

An IL-6 deficiency can, for example, increase inflammatory bone destruction (Balto, K., et al., 2001. Infection and immunity, 69(2), pp. 744-750) and/or exacerbate Huntington's disease (Wertz, M. H., et al., 2020. Molecular neurodegeneration, 15, pp. 1-8). Exemplary diseases associated with IL-6 deficiency are described in Murakami, et al., 2019. Immunity, 50(4), pp. 812-831.

In one aspect, the invention provides a Bifidobacterium lactis supernatant for use in treating and/or preventing an IL-6 mediated disease. In another aspect, the invention provides for use of a Bifidobacterium lactis supernatant for the manufacture of a medicament for treating and/or preventing an IL-6 mediated disease. In another aspect, the invention provides a method of treating and/or preventing an IL-6 mediated disease in a subject, comprising administering a Bifidobacterium lactis supernatant to the subject.

In one aspect, the invention provides a supplement or nutritional composition comprising a Bifidobacterium lactis supernatant for use in treating and/or preventing IL-6 mediated disease. In another aspect, the invention provides a supplement or nutritional composition comprising a Bifidobacterium lactis supernatant for the manufacture of a medicament for treating and/or preventing an IL-6 mediated disease. In another aspect, the invention provides a method of treating and/or preventing an IL-6 mediated disease in a subject, comprising administering a supplement or nutritional composition comprising a Bifidobacterium lactis supernatant to the subject.

### Methods of Manufacture

The Bifidobacterium lactis supernatant of the present invention may be prepared by any suitable method known in the art. For example, preparation of bacterial supernatants is described in Moradi, M., et al., 2021. Enzyme and Microbial Technology, 143, p. 109722.

Exemplary culture conditions and processing steps are described above in the section entitled “Supernatant preparation”, and the methods of manufacture according to the present invention may include any of the steps described therein.

In one aspect, the present invention provides a method of manufacturing a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant, preferably wherein the Bifidobacterium lactis cells are removed by centrifugation; and
(c) optionally, pasteurising the Bifidobacterium lactis supernatant.

The method may comprise any other suitable processing steps. In preferred embodiments, the method further comprises a step (d) of drying the Bifidobacterium lactis supernatant.

The Bifidobacterium lactis supernatant may be any Bifidobacterium lactis supernatant described herein.

In one aspect, the present invention provides a method of providing a supplement comprising a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant, preferably wherein the Bifidobacterium lactis cells are removed by centrifugation;
(c) optionally, pasteurising the Bifidobacterium lactis supernatant;
(d) drying the Bifidobacterium lactis supernatant to provide a Bifidobacterium lactis supernatant powder; and
(e) processing the Bifidobacterium lactis supernatant powder to provide a supplement comprising the Bifidobacterium lactis supernatant powder.

The method may comprise any other suitable processing steps. For example, in some embodiments, step (e) comprises a step of encapsulating, compressing and/or packaging the Bifidobacterium lactis supernatant powder to provide the supplement. For example, in some embodiments step (e) comprises a step of encapsulating the Bifidobacterium lactis supernatant powder to provide a capsule comprising the Bifidobacterium lactis supernatant.

The supplement may be any supplement described herein.

Exemplary culture conditions and processing steps are described above in the section entitled “Supernatant preparation”, and the methods of manufacture according to the present invention may include any of the steps described therein.

### EXAMPLES

The invention will now be further described by way of Examples, which are meant to serve to assist one of ordinary skill in the art in carrying out the invention and are not intended in any way to limit the scope of the invention.

### Example 1—Immune Effect of B. lactis Supernatant Versus B. lactis Probiotic Versus a Combination of Both

### Production of Supernatant Powder

Supernatant of B. lactis (Bifidobacterium animalis subsp. lactis CNCM I-3446) was produced at pilot plant scale (8000 L main fermentation). The inoculum for the main fermentation was produced at pilot plant scale in a 1200 L starter fermentation. The main fermentation step was carried out at 37° C. with pH control (pH 6.0), stirring and head space gassing (carbon dioxide) until stationary phase was reached. The media contained yeast extract, sodium ascorbate and dextrose. After reaching stationary phase, the fermentate was centrifuged with a continuous centrifuge to remove the vast majority of the bacterial cells. Subsequently, the supernatant was pasteurized with a plate heat exchanger at 99° C. for 10 seconds to inactivate the remaining live cells before filling into canisters and freezing.

To convert the liquid supernatant into a powder that could be used for example in supplements, a drying process was developed. Spray drying was selected as preferred technology as this technology is suitable for large volumes, it is energy efficient and readily available. For spray drying experiments a table top Büchi spray dryer was used. Carrier materials were tested that would reduce stickiness and improve physical stability. Suitable food grade carrier materials identified in these tests were oat fibre, maltodextrin, acacia gum (gum arabic), starch and inulin. The carriers were added to the supernatant solutions with total solids ratios of 1:3 and 1:1 (carrier: supernatant dry solids). Overall the ratio of 1:1 resulted in less sticky behaviour compared to the 1:3 ratio.

Out of all the powders produced the supernatant powder with acacia gum with 1:1 ratio was selected for an in vitro colon simulation study, followed by an in vitro immune assay.

### Short-Term Colonic Incubations

To mimic intestinal conversion of the supernatant powder with or without the probiotic B. lactis (Bifidobacterium animalis subsp. lactis CNCM I-3446) in an adult intestinal microbiota, an in vitro model of the colon was employed. Short-term batch experiments represent a simplified simulation of the continuous Simulator of the Human Microbial Ecosystem (SHIME®), a model which has been used for over 20 years and has been validated with in vivo parameters. Short-term colonic incubation assays typically consist of a colonic fermentation of a selected dose of the test compound(s) under simulated conditions representative for the proximal large intestine of a healthy adult, using a bacterial inoculum obtained from selected donors.

Freshly prepared human faecal samples were used as a source of the microbial community for inoculation of the colonic models. Faecal inocula were obtained from three different healthy donors (donors D1, D2, D3). At the start of the short-term colonic incubations, the test products were added to sugar-depleted nutritional medium containing basal nutrients that are present in the colon (e.g. host-derived glycans such as mucin). The dose for B. lactis supernatant powder was 3.6 g/L and for B. lactis probiotic culture powder 1.4E+08 cfu/mL (normal dose). The two ingredients were used individually and in combination. Furthermore, a low dose combination was tested with 2.6 g/L supernatant powder and 1.4E+07 cfu/mL B. lactis. A blank was also included, containing only sugar-depleted nutritional medium to mimic the background activity of the community. Incubations were performed during 48 h, at 37° C., under shaking (90 rpm) and anaerobic conditions. In order to account for biological variability, all tests were performed in triplicate. After the 48h colonic incubations of the faecal inoculum of the three donors with the test ingredients in sugar-depleted nutritional medium, samples were taken and cleaned of cells by sterile filtration (0.22 μm). These sterile filtered samples were then used in the immune assay.

### Immune Assay

To mimic the interface between host and gut microbiome, in vitro models have been developed which are based on intestinal epithelial-like cells and immune cells of human origin. In this study, a co-culture model of intestinal epithelial-like cells (Caco-2 cells) and human monocytes/macrophages (THP1 cells) (Possemiers, S., et al. (2013) J. Agric. Food Chem., 61: 9380-939) was used, based on the work of Satsu and colleagues (Satsu, H., et al. (2006) Exp. Cell Res., 312: 3909-939). In this model, anti-inflammatory potential is determined via analysis of the cytokine profile (increase in anti-inflammatory cytokines and decrease in pro-inflammatory cytokines). The filter-sterilized colonic suspensions collected from the colonic incubations are brought in contact with the apical side of the co-cultures (Caco-2 cells). The effects observed on the basolateral chamber (where the THP1 cells reside) are then mediated indirectly by signals produced by the Caco-2 cells and/or by the transport of micro- and macro-molecules. This allows to evaluate the effect induced by the product and the fermentation derived metabolites produced by the gut microbiota during the digestive steps (so not by the pure product).

The co-culture experiment was performed as previously described (Daguet, D., et al. (2016) Journal of Functional Foods, 20: 369-379). Briefly, Caco-2 cells (HTB-37; American Type Culture Collection) were seeded in 24-well semi-permeable inserts. Caco-2 monolayers were cultured for 14 days, with three medium changes/week, until a functional cell monolayer was obtained. THP1-Blue™ cells were seeded in 24-well plates and treated with phorbol 12-myristate 13-acetate (PMA) that induces the differentiation of the cells into macrophage-like cells, which are able to adhere and are primed for toll-like receptor (TLR) signalling. Caco-2-bearing inserts were placed on top of the PMA-differentiated THP1-Blue™ cells for further experiments, as previously described (Possemiers, S., et al. (2013) J. Agric. Food Chem., 61: 9380-939; Daguet, D., et al. (2016) Journal of Functional Foods, 20: 369-379). Briefly, the apical compartment (containing the Caco-2 cells) was filled with sterile-filtered (0.22 μm) colonic batch suspensions. Cells were also treated apically with sodium butyrate (NaB) (Sigma-Aldrich) as positive control. The basolateral compartment (containing the THP1-Blue™ cells) was filled with Caco-2 complete medium. Cells were also exposed to Caco-2 complete medium in both chambers as control.

Cells were treated for 24h, then the basolateral supernatant was discarded and cells were stimulated at the basolateral side with Caco-2 complete medium containing ultrapure lipopolysaccharide (LPS). Cells were also stimulated at the basolateral side with LPS in combination with hydrocortisone (HC) (Sigma-Aldrich) and medium without LPS (LPS−) as controls for 6 h. After LPS stimulation, the basolateral supernatants were collected for cytokine measurement (human IL-1β, IL-6, IL-8, IL-10, TNF-α, CXCL10 and MCP-1 by Luminex® multiplex (Affymetrix-eBioscience)) and for NF-κB activity, according to the manufacturers' instructions. MCP-1 was excluded as the values fell outside of the upper limit of the standard and so results are not reliable.

All treatments were done in triplicate. Cells were incubated at 37° C. in a humidified atmosphere of air/C02 (95:5, v/v). All colonic batch samples were taken as biological replicates (n=3) in the cell assay. To evaluate differences in immune markers, treatment samples were compared to their respective control samples using a two-way ANOVA with Dunnett's multiple comparisons test and are represented by (*). (*), (**), (***) and (****) representing p<0.05, P<0.01, p<0.001 and p<0.0001, respectively. All statistics were performed using GraphPad Prism version 8.3.0 for Windows (GraphPad Software, San Diego, CA, USA).

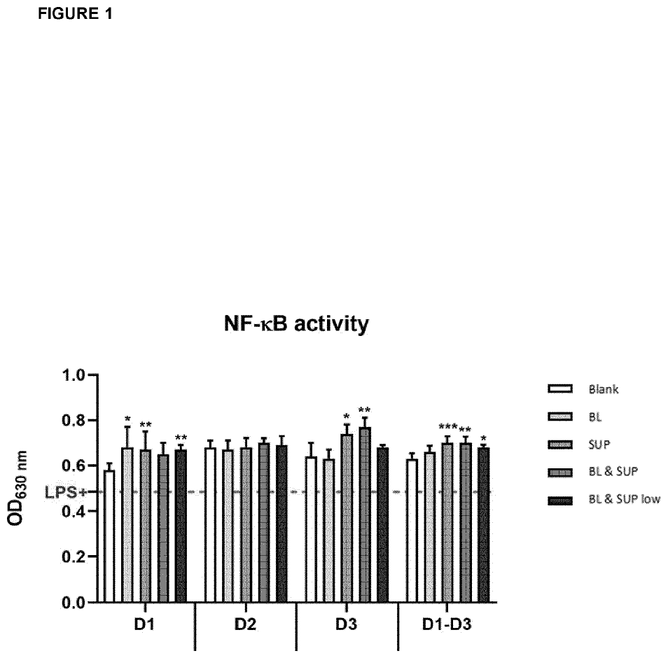

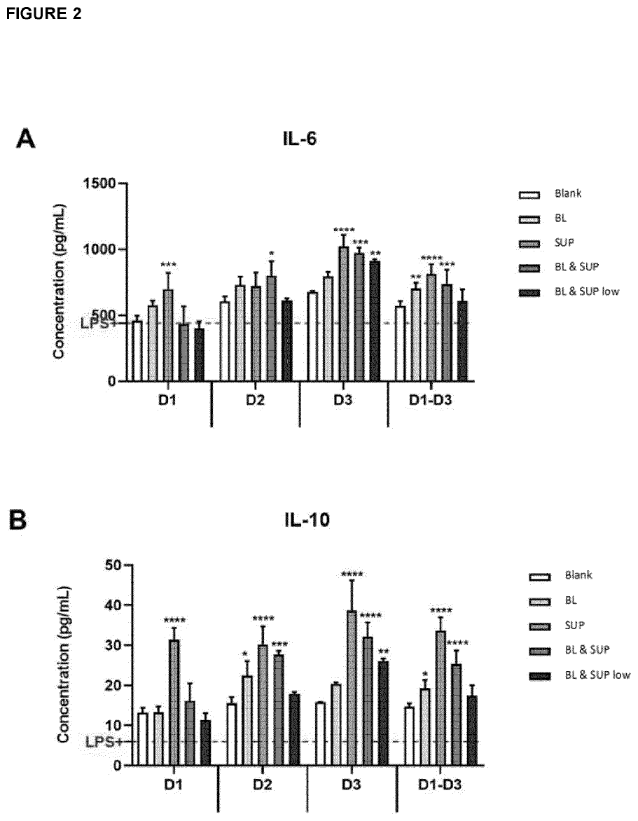

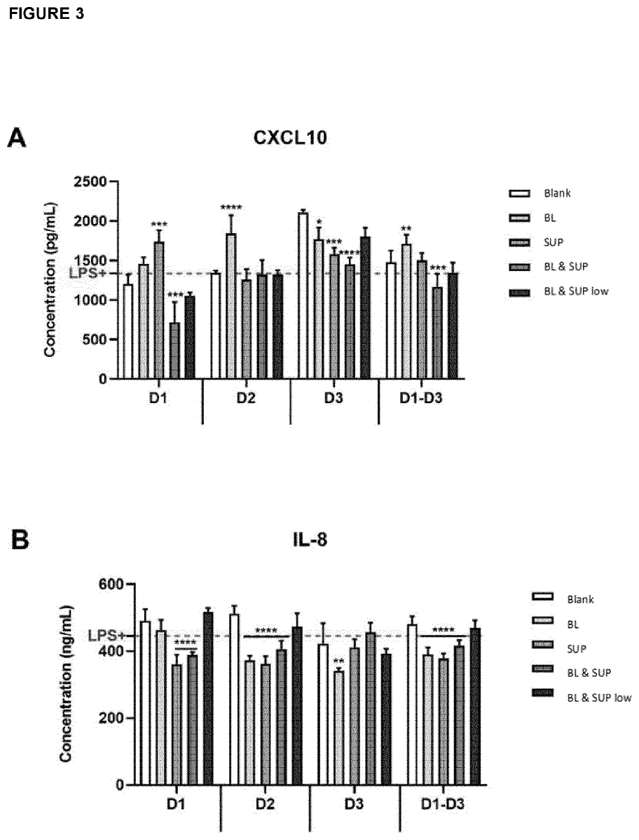

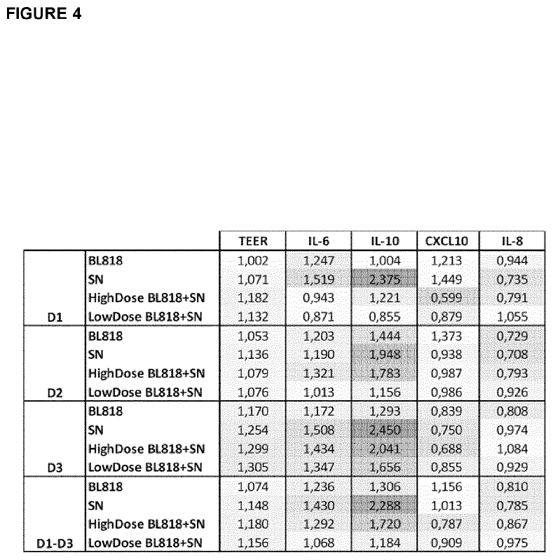

All colonic batch suspensions increased the NF-κB activity compared to the LPS+control (FIG. 1). Furthermore, in donor 1, incubation with B. lactis significantly increased the NF-κB activity compared to its control. In addition, in donors 1 and 3, B. lactis derived supernatant significantly increased the NF-κB activity; as well as the combination of B. lactis with its supernatant. These effects were reflected when looking at the average of all donors. To conclude, B. lactis-derived supernatant and the combination of B. lactis with its supernatant significantly increased the LPS-induced NF-κB activity in 2 out of 3 donors.

All colonic batch suspensions (except for the blank and B. lactis & supernatant samples in donor 1) increased the secretion of the anti-inflammatory cytokine IL-6 compared to the LPS+ control (FIG. 2A). Moreover, B. lactis tended to increase the LPS-induced IL-6 secretion in all donors, while the supernatant significantly increased the IL-6 secretion in donors 1 and 3. In donor 2, the supernatant tended to increase the IL-6 secretion. At both concentrations, the combination of B. lactis & supernatant significantly increased the IL-6 secretion in donor 3. In donor 2, only the normal dose of B. lactis & supernatant significantly increased IL-6 secretion, while no effects were seen in donor 1. When looking at the average of all donors, B. lactis, its supernatant and the combination of B. lactis & supernatant (normal dose) significantly increased the LPS-induced IL-6 secretion. In general, the effects on IL-6 secretion were most pronounced for the supernatant.

All colonic batch suspensions increased the secretion of the anti-inflammatory cytokine IL-10 compared to the LPS+ control (FIG. 2B). Incubation with B. lactis increased the secretion of IL-10 in donors 2 and 3, reaching significance in donor 2. Furthermore, the supernatant significantly increased the IL-10 secretion in all donors. At both concentrations, the combination of B. lactis & supernatant significantly increased the secretion of IL-10 in donor 3; while in donor 2; this effect was only observed at the normal dose level. When looking at the average of all donors, B. lactis, its supernatant and the combination of B. lactis & supernatant (normal dose) significantly increased the LPS-induced IL-10 secretion. In general, the effects on IL-10 secretion were most pronounced for the supernatant.

To conclude, B. lactis slightly increased the secretion of the anti-inflammatory cytokines IL-6 and IL-10. In contrast, B. lactis-derived supernatant strongly increased the secretion of these cytokines. Finally, at the normal dose, B. lactis & supernatant also significantly increased the secretion of IL-6 and IL-10; although to a lesser extent than the supernatant alone.

The combination of B. lactis & supernatant (normal dose) significantly decreased the LPS-induced secretion of the chemokine CXCL10 in donors 1 and 3 compared to the control (FIG. 3A). Further, B. lactis-derived supernatant significantly reduced the CXCL10 secretion in donor 3. B. lactis treated colonic suspensions slightly decreased the secretion of CXCL10 in donor 3; while it increased its secretion in donor 2. When looking at the average of all donors, the combination of B. lactis & supernatant (normal dose) significantly decreased the expression of CXCL10 compared to the control. CXCL10 levels for B. lactis-treatment samples of donor 2 and blank samples of donor 3 could not be exactly determined as some of the replicate values fell out of the linear range of the standard. Therefore, concentrations might be overestimated in these samples.

B. lactis-treated colonic suspensions significantly decreased the secretion of the chemokine IL-8 in donors 2 and 3, compared to the control (FIG. 3B). In addition, B. lactis-derived supernatant and the combination of B. lactis+supernatant (normal dose) significantly decreased the IL-8 secretion in donors 1 and 2. When looking at the average of all donors, B. lactis, its supernatant and the combination of B. lactis & supernatant (normal dose) significantly decreased the IL-8 secretion.

In conclusion, B. lactis, its supernatant and the combination of B. lactis & supernatant (normal dose) significantly reduced the secretion of the chemokine IL-8. In addition, the normal dose of B. lactis & supernatant significantly reduced the secretion of the chemokine CXCL10.

In order to provide an overview of the changes induced by the different treatments comparatively to the blank control, the treatment colonic batch suspensions were normalized to the control colonic batch suspensions and summarized in FIG. 4. All treatments increased the secretion of the anti-inflammatory cytokines IL-6 and IL-10. The strongest effects were noted for the B. lactis-derived supernatant, followed by the combination of B. lactis probiotic and its supernatant at the normal dose. Moreover, all treatments decreased the secretion of the chemokine IL-8, with the strongest effect observed for the supernatant. In contrast, the combination of B. lactis and its supernatant (normal dose) showed the strongest inhibitory effect on secretion of the chemokine CXCL10.

To conclude, B. lactis-derived supernatant and the combination of B. lactis and its supernatant (normal dose) demonstrated significant anti-inflammatory properties in the in vitro Caco-2/THP1 co-culture model used. It was surprising that the supernatant was in several aspects superior to the probiotic Bifidobacterium animalis subsp. lactis CNCM I-3446, with the probiotic being well-documented for its immuno-modulatory effects in various clinical trials.

### Embodiments

Various preferred features and embodiments of the present invention will now be described with reference to the following numbered paragraphs (paras).

1. A Bifidobacterium lactis supernatant for use in enhancing expression of anti-inflammatory cytokines and/or reducing expression of pro-inflammatory chemokines in the gastrointestinal tract of a subject having or at risk of an overactive immune system disorder.

2. The Bifidobacterium lactis supernatant for use according to para 1, wherein the Bifidobacterium lactis supernatant enhances expression of IL-10 in the gastrointestinal tract of the subject.

3. The Bifidobacterium lactis supernatant for use according to para 1 or 2, wherein the overactive immune system disorder is an IL-10 mediated disease.

4. A Bifidobacterium lactis supernatant for use in enhancing expression of IL-10 in the gastrointestinal tract of a subject having or at risk of an IL-10 mediated disease.

5. A Bifidobacterium lactis supernatant for use in treating or preventing an IL-10 mediated disease by enhancing expression of IL-10 in the gastrointestinal tract of a subject.

6. A Bifidobacterium lactis supernatant for use in enhancing expression of IL-6 in the gastrointestinal tract of a subject having or at risk of an IL-6 mediated disease.

7. A Bifidobacterium lactis supernatant for use in treating or preventing an IL-6 mediated disease by enhancing expression of IL-6 in the gastrointestinal tract of a subject.

8. The Bifidobacterium lactis supernatant for use according to any preceding para, wherein the Bifidobacterium lactis is selected from: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

9. The Bifidobacterium lactis supernatant for use according to any preceding para, wherein the Bifidobacterium lactis is a Bifidobacterium animalis subsp. lactis having at least

99.0%, at least 99.1%, at least 99.2%, at least 99.3%, at least 99.4%, at least 99.5%, at least 99.6%, at least 99.7%, at least 99.8%, or at least 99.9% sequence identity to Bifidobacterium animalis subsp. lactis CNCM I-3446.

10. The Bifidobacterium lactis supernatant for use according to any preceding para, wherein the Bifidobacterium lactis is Bifidobacterium animalis subsp. lactis CNCM I-3446.

11. The Bifidobacterium lactis supernatant for use according to any preceding para, wherein the Bifidobacterium lactis supernatant is obtained or obtainable by culturing Bifidobacterium lactis in a culture media comprising sugar and yeast extract, and, optionally, sodium ascorbate and/or polysorbate.

12. The Bifidobacterium lactis supernatant for use according to para 11, wherein:
(i) the culture media comprises about 1 wt % to about 6 wt %, or about 2 wt % to about 4 wt % sugar;
(ii) the culture media comprises about 1 wt % to about 10 wt %, or about 1 wt % to about 6 wt %, or about 2 wt % to about 4 wt % yeast extract;
(iii) the culture media comprises about 0 wt % to about 0.5 wt %, or about 0.1 wt % to about 0.2 wt % sodium ascorbate; and
(iv) the culture media comprises about 0 wt % to about 1 wt %, or about 0 wt % to about 0.3 wt % polysorbate.

13. The Bifidobacterium lactis supernatant for use according to para 11 or 12, wherein the sugar is glucose, dextrose and/or glucose syrup.

14. The Bifidobacterium lactis supernatant for use according to any of paras 11 to 13, wherein the Bifidobacterium lactis supernatant is obtained or obtainable by culturing at a pH of from about 5 to about 7, a pH of from about 5.5 to about 6.5, or a pH of about 6.

15. The Bifidobacterium lactis supernatant for use according to any preceding para, wherein the Bifidobacterium lactis supernatant is obtained or obtainable by culturing the Bifidobacterium lactis until stationary phase is reached.

16. The Bifidobacterium lactis supernatant for use according to any preceding para, wherein the Bifidobacterium lactis supernatant is obtained or obtainable by culturing the Bifidobacterium lactis under anaerobic conditions.

17. The Bifidobacterium lactis supernatant for use according to any preceding para, wherein the Bifidobacterium lactis supernatant is obtained or obtainable by removing substantially all the Bifidobacterium lactis cells from a Bifidobacterium lactis fermentate.

18. The Bifidobacterium lactis supernatant for use according to any preceding para, wherein the Bifidobacterium lactis supernatant is pasteurised.

19. The Bifidobacterium lactis supernatant for use according to any preceding para, wherein the Bifidobacterium lactis supernatant is dried.

20. The Bifidobacterium lactis supernatant for use according to para 19, wherein the Bifidobacterium lactis supernatant is dried by spray-drying.

21. The Bifidobacterium lactis supernatant for use according to para 20, wherein the Bifidobacterium lactis supernatant is spray-dried with a carrier material selected from one or more of: oat fibre, maltodextrin, acacia gum, starch and inulin, preferably wherein the Bifidobacterium lactis supernatant is spray-dried with acacia gum

22. The Bifidobacterium lactis supernatant for use according to para 21, wherein the Bifidobacterium lactis supernatant and carrier material are mixed in a total solids ratio of from about 1:3 to about 2:1 (carrier:supernatant dry solids), preferably wherein the total solids ratio is about 1:1 (carrier:supernatant dry solids).

23. The Bifidobacterium lactis supernatant for use according to any preceding para, wherein the Bifidobacterium lactis supernatant is administered orally.

24. The Bifidobacterium lactis supernatant for use according to any preceding para, wherein the Bifidobacterium lactis supernatant is in the form of a supplement or a nutritional composition.

25. The Bifidobacterium lactis supernatant for use according to any preceding para, wherein the Bifidobacterium lactis supernatant is in the form of a capsule or a tablet.

26. The Bifidobacterium lactis supernatant for use according to any preceding para, wherein compared to Bifidobacterium lactis culture media the Bifidobacterium lactis supernatant has:
(i) a decreased concentration of total sugar;
(ii) an increased concentration of total acids; and/or
(iii) a decreased concentration of total amino acids.

27. The Bifidobacterium lactis supernatant for use according to para 26, wherein compared to Bifidobacterium lactis culture media the concentration of total sugar has decreased by at least about 50%, at least about 60%, at least about 70%, at least about 80%, or at least about 90%.

28. The Bifidobacterium lactis supernatant for use according to para 26 or 27, wherein compared to Bifidobacterium lactis culture media the concentration of total acids has increased by at least about 70%, by at least about 80%, or by at least about 90% of the concentration decrease in total sugars.

29. The Bifidobacterium lactis supernatant for use according to any of paras 26 to 28, wherein compared to Bifidobacterium lactis culture media the concentration of total amino acids has decreased by at least about 0.1 wt %, by at least about 0.2 wt %, or by at least about 0.3 wt %.

30. The Bifidobacterium lactis supernatant for use according to any preceding para, wherein before pasteurisation the Bifidobacterium lactis supernatant has a viable cell count of from about 1×10⁷ to about 1×10⁹ cfu/ml.

31. The Bifidobacterium lactis supernatant for use according to any preceding para, wherein the Bifidobacterium lactis supernatant comprises about 4 wt % or less, about 3 wt % or less, about 2 wt % or less, about 1 wt % or less, or about 0.5 wt % or less total sugars.

32. The Bifidobacterium lactis supernatant for use according to any preceding para, wherein the Bifidobacterium lactis supernatant comprises about 0.5 wt % or more, about 1 wt % or more, about 1.5 wt % or more, or about 2 wt % or more total acids.

33. The Bifidobacterium lactis supernatant for use according to any preceding para, wherein the Bifidobacterium lactis supernatant comprises about 3.5 wt % or less, about 2 wt % or less, about 1 wt % or less, about 0.8 wt % or less, or about 0.6 wt % or less total amino acids.

34. The Bifidobacterium lactis supernatant for use according to any preceding para, wherein the Bifidobacterium lactis supernatant has a pH of from about 5 to about 7, from about 5.5. to about 6.5, or of about 6, preferably wherein the Bifidobacterium lactis supernatant has a pH of about 6.2.

35. The Bifidobacterium lactis supernatant for use according to any preceding para, wherein the Bifidobacterium lactis supernatant is used in combination with one or more probiotics, prebiotics, or synbiotics, preferably wherein the Bifidobacterium lactis supernatant is used in combination with one or more probiotics.

36. The Bifidobacterium lactis supernatant for use according to any preceding para, wherein the Bifidobacterium lactis supernatant is used in combination with a Bifidobacterium lactis probiotic.

37. The Bifidobacterium lactis supernatant for use according to para 36, wherein the probiotic Bifidobacterium lactis is the same as the Bifidobacterium lactis from which the supernatant is derived.

38. The Bifidobacterium lactis supernatant for use according to any preceding para, wherein the subject has or is at risk of an IL-10 deficiency.

39. The Bifidobacterium lactis supernatant for use according to any preceding para, wherein the subject is a mammal, preferably wherein the subject is a human, a dog, a cat, a rodent, or a rabbit, more preferably wherein the subject is human.

40. The Bifidobacterium lactis supernatant for use according to any of paras 3 to 5 or 8 to 39, wherein the IL-10 mediated disease is selected from: an inflammatory bowel disease (IBD), an allergic disease, dermatitis, an autoimmune disease, an infection-associated immunopathology, colorectal cancer, impaired bone healing, and atherosclerosis.

41. The Bifidobacterium lactis supernatant for use according to para 40, wherein:
(i) the IBD is Crohn's disease (CD) or ulcerative colitis (UC);
(ii) the allergic disease is allergic asthma or a food allergy;
(iii) the dermatitis is atopic dermatitis or contact dermatitis;
(iv) the autoimmune disease is selected from: systemic lupus erythematosus, rheumatoid arthritis, multiple sclerosis, psoriasis, ankylosing spondylitis, and Guillian-Barre syndrome; and/or
(v) the infection is selected from: a protozoan infection, a bacterial infection, a nematode infection, a viral infection, and a fungal infection.

42. A Bifidobacterium lactis supernatant, wherein the Bifidobacterium lactis supernatant has been spray-dried.

43. The Bifidobacterium lactis supernatant according to para 42, wherein the Bifidobacterium lactis supernatant is as defined according to any of paras 8 to 19 or 21 to 34.

44. A supplement comprising a Bifidobacterium lactis supernatant, preferably wherein the supplement is in the form of a capsule or a tablet.

45. A nutritional composition comprising a Bifidobacterium lactis supernatant.

46. The supplement according to para 44, or the nutritional composition according to para

45, wherein the Bifidobacterium lactis supernatant is as defined according to any of paras 8 to 23 or 25 to 34.

47. The supplement according to para 44 or 46, or the nutritional composition according to para 45 or 46 wherein the supplement or nutritional composition comprises one or more probiotics, prebiotics, or synbiotics, preferably wherein the supplement or nutritional composition comprises one or more probiotics.

48. The supplement according to any of paras 44, 46 or 47, or the nutritional composition according to any of paras 45 to 47, wherein the supplement or nutritional composition comprises a Bifidobacterium lactis probiotic, preferably wherein the probiotic Bifidobacterium lactis is the same as the Bifidobacterium lactis from which the supernatant is derived.

49. A method of manufacturing a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant; and
(c) optionally, pasteurising the Bifidobacterium lactis supernatant.

50. The method according to para 49, wherein the method further comprises a step (d) of drying the Bifidobacterium lactis supernatant, preferably wherein the Bifidobacterium lactis supernatant is dried by spray-drying.

51. The method according to para 49 or 50, wherein the Bifidobacterium lactis supernatant is a Bifidobacterium lactis supernatant according to para 42 or 43.

52. A method of manufacturing a supplement comprising a Bifidobacterium lactis supernatant, the method comprising:
(a) culturing a Bifidobacterium lactis in a culture media to provide a Bifidobacterium lactis fermentate;
(b) removing substantially all the Bifidobacterium lactis cells from the Bifidobacterium lactis fermentate to provide a Bifidobacterium lactis supernatant;
(c) optionally, pasteurising the Bifidobacterium lactis supernatant;
(d) drying the Bifidobacterium lactis supernatant to provide a Bifidobacterium lactis supernatant powder; and
(e) encapsulating, compressing and/or packaging the Bifidobacterium lactis supernatant powder to provide a supplement comprising the Bifidobacterium lactis supernatant.

53. The method according to para 52, wherein the Bifidobacterium lactis supernatant is dried by spray-drying.

54. The method according to para 52 or 53, wherein the Bifidobacterium lactis supernatant powder is encapsulated to provide a capsule comprising the Bifidobacterium lactis supernatant.

55. The method according to any of paras 52 to 54, wherein the supplement is a supplement according to any of paras 44, 46, 47, or 48.

1. A method for enhancing expression of anti-inflammatory cytokines and/or reducing expression of pro-inflammatory chemokines in the gastrointestinal tract of a subject having or at risk of an overactive immune system disorder comprising administering a Bifidobacterium lactis supernatant to the subject.

2. A method for use in enhancing expression of IL-10 in the gastrointestinal tract of a subject having or at risk of an IL-10 mediated disease comprising administering a Bifidobacterium lactis supernatant to the subject.

3. A method for use in treating or preventing an IL-10 mediated disease by enhancing expression of IL-10 in the gastrointestinal tract of a subject comprising administering a Bifidobacterium lactis supernatant to the subject.

4. Method according to claim 1, wherein the Bifidobacterium lactis is selected from the group consisting of: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

5. The method according to claim 1, wherein the Bifidobacterium lactis supernatant is obtained or obtainable by culturing Bifidobacterium lactis in a culture media comprising sugar and yeast extract, wherein:
(i) the culture media comprises about 1 wt % to about 6 wt % sugar;
(ii) the culture media comprises about 1 wt % to about 10 wt % yeast extract;
(iii) the culture media comprises about 0 wt % to about 0.5 wt % sodium ascorbate; and
(iv) the culture media comprises about 0 wt % polysorbate.

6-9. (canceled)

10. The method according to claim 1, wherein compared to Bifidobacterium lactis culture media the Bifidobacterium lactis supernatant has:
(i) a decreased concentration of total sugar;
(ii) an increased concentration of total acids; and
(iii) a decreased concentration of total amino acids.

11. The method according to claim 1, wherein the Bifidobacterium lactis supernatant comprises: (i) about 4 wt % or less less total sugars; (ii) about 0.5 wt % or more total acids; and (iii) about 3.5 wt % less total amino acids.

12. The method according to claim 1, wherein the Bifidobacterium lactis supernatant is used in combination with one or more probiotics, prebiotics, or synbiotics.

13. (canceled)

14. The method according to claim 3, wherein the IL-10 mediated disease is selected from the group consisting of: an inflammatory bowel disease (IBD), an allergic disease, dermatitis, an autoimmune disease, an infection-associated immunopathology, colorectal cancer, impaired bone healing, and atherosclerosis.

15-16. (canceled)

17. Method according to claim 2, wherein the Bifidobacterium lactis is selected from the group consisting of: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

18. Method according to claim 3, wherein the Bifidobacterium lactis is selected from the group consisting of: Bifidobacterium animalis subsp. lactis CNCM I-3446, Bifidobacterium animalis subsp. lactis BI12, Bifidobacterium animalis subsp. lactis BLC1, Bifidobacterium animalis subsp. lactis DSM10140, Bifidobacterium animalis subsp. lactis V9, Bifidobacterium animalis subsp. lactis BI-04, Bifidobacterium animalis subsp. lactis Bi-07, Bifidobacterium animalis subsp. lactis B420, Bifidobacterium animalis subsp. lactis BB-12, Bifidobacterium animalis subsp. lactis AD011, Bifidobacterium animalis subsp. lactis HN019, Bifidobacterium animalis subsp. lactis DN-173 010, Bifidobacterium animalis subsp. lactis ATCC 27536, and Bifidobacterium animalis subsp. lactis VTT E-012010.

19. The method according to claim 2, wherein the Bifidobacterium lactis supernatant is obtained or obtainable by culturing Bifidobacterium lactis in a culture media comprising sugar and yeast extract, wherein:
(i) the culture media comprises about 1 wt % to about 6 wt % sugar;
(ii) the culture media comprises about 1 wt % to about 10 wt % yeast extract;
(iii) the culture media comprises about 0 wt % to about 0.5 wt % sodium ascorbate; and
(iv) the culture media comprises about 0 wt % to about 1 wt % polysorbate.

20. The method according to claim 3, wherein the Bifidobacterium lactis supernatant is obtained or obtainable by culturing Bifidobacterium lactis in a culture media comprising sugar and yeast extract, wherein:
(i) the culture media comprises about 1 wt % to about 6 wt % sugar;
(ii) the culture media comprises about 1 wt % to about 10 wt % yeast extract;
(iii) the culture media comprises about 0 wt % to about 0.5 wt % sodium ascorbate; and
(iv) the culture media comprises about 0 wt % to about 1 wt % polysorbate.

21. The method according to claim 2, wherein compared to Bifidobacterium lactis culture media the Bifidobacterium lactis supernatant has:
(i) a decreased concentration of total sugar;
(ii) an increased concentration of total acids; and
(iii) a decreased concentration of total amino acids.

22. The method according to claim 3, wherein compared to Bifidobacterium lactis culture media the Bifidobacterium lactis supernatant has:
(i) a decreased concentration of total sugar;
(ii) an increased concentration of total acids; and
(iii) a decreased concentration of total amino acids.

23. The method according to claim 2, wherein the Bifidobacterium lactis supernatant comprises: (i) about 4 wt % or less total sugars; (ii) about 0.5 wt % or more total acids; and (iii) about 3.5 wt % less total amino acids.

24. The method according to claim 3, wherein the Bifidobacterium lactis supernatant comprises: (i) about 4 wt % or less total sugars; (ii) about 0.5 wt % or more total acids; and (iii) about 3.5 wt % less total amino acids.

25. The method according to claim 2, wherein the Bifidobacterium lactis supernatant is used in combination with one or more probiotics, prebiotics, or synbiotics.

26. The method according to claim 3, wherein the Bifidobacterium lactis supernatant is used in combination with one or more probiotics, prebiotics, or synbiotics.
